SEQUENCE LISTING (1 sequence)

SEQ ID NO: 1 - protein, 191 aa, Homo sapiens [sequence omitted]

(19) United States
(12) Patent Application Publication
(10) Pub. No.: US 2015/0250882 A1
(43) Pub. Date: Sep. 10, 2015

(54) Fatty Acid Acylated Amino Acids for Growth Hormone Delivery

(71) Applicant: Novo Nordisk Health Care AG, Zürich (CH)

(72) Inventors: Mats Reslow, Lund (SE); Leif Noerskov-Lauritsen, Tappernoeje (DK); Heidi Westh Bagger, Koebenhavn S (DK); Christine Bruun Schioedt, Broenshoej (DK)

(21) Appl. No.: 14/433,181

(22) PCT Filed: Oct. 17, 2013
(86) PCT No.: PCT/EP2013/071719

§ 371 Date: Apr. 2, 2015

Related U.S. Application Data
(60) Provisional application No. 61/714,982, filed on Oct. 17, 2012.

(30) Foreign Application Priority Data
Oct. 17, 2012 (EP) 12188838.2

(51) Int. Cl. A61K 47/22; A61K 47/18; A61K 38/27

(52) U.S. Cl. CPC A61K 47/22; A61K 38/27; A61K 47/183

(57) ABSTRACT

The present invention relates to growth hormone compositions comprising a fatty acid acylated amino acids (FA-aa's), which may be used in pharmaceutical compositions such as pharmaceutical compositions for oral administration of growth hormone.

### TECHNICAL FIELD

The technical field of this invention relates to fatty acid acylated amino acids (FA-aa's) for oral delivery of therapeutic growth hormone compounds and pharmaceutical compositions comprising such FA-aa's.

### BACKGROUND

Many pathological states due to deficiencies in or complete failure of the production of a certain macromolecules (e.g. proteins and peptides) are treated with an invasive and inconvenient parenteral administration of therapeutic macromolecules, such as hydrophilic peptides or proteins. One example hereof is the administration of human growth hormone (hGH) for the treatment of growth hormone deficiencies or disease or disorders where the patient benefits from an increased in the amount of circulating growth hormone, usually by once daily doses of hGH. Compared hereto, an orally administered medicament is desirable due to its non-invasive nature and has a great potential to decrease the patient's discomfort related to drug administration and to increase drug compliance. However several barriers exist; such as the enzymatic degradation in the gastrointestinal (GI) tract, drug efflux pumps, insufficient and variable absorption from the intestinal mucosa, as well as first pass metabolism in the liver and until now no products for oral delivery of therapeutic hydrophilic proteins are found to be marketed.

Growth hormone (GH) is a polypeptide hormone secreted by the anterior pituitary in mammals. Dependent on species GH is a protein composed of approximately 190 amino acid residues corresponding to a molecular weight of approximately 22 kDa. GH binds to and signals through cell surface receptors, the GH receptors (GHR). GH plays a key role in promoting growth, maintaining normal body composition, anabolism and lipid metabolism. It also has direct effects on intermediate metabolism, such as decreased glucose uptake, increased lipolysis and increased amino acid uptake and protein synthesis. The hormone also exerts effects on other tissues including adipose tissue, liver, intestine, kidney, skeleton, connective tissue and muscle. Recombinant hGH has been produced and commercially available as, for ex: Genotropin™ (Pharmacia Upjohn), Nutropin™ and Protropin™ (Genentech), Humatrope™ (Eli Lilly), Serostim™ (Serono), Norditropin (Novo Nordisk), Omnitrope (Sandoz), Nutropin Depot (Genentech and Alkermes). Additionally, an variant with an additional methionine residue at the N-terminal end is also marketed as, for ex: Somatonorm™ (Pharmacia Upjohn/Pfizer).

Human growth hormone is degraded by various digestive enzymes found in the stomach (pepsin), in the intestinal lumen (chymotrypsin, trypsin, elastase, carboxypeptidases, etc.) and in the mucosal surfaces of the GI tract (aminopeptidases, carboxypeptidases, enteropeptidases, dipeptidyl peptidases, endopeptidases, etc.).

Various routs for obtaining a protease stabilized human growth hormone compound have been explored and examples of such molecules are described in WO 2011/089250 and WO2011/089255. A subsequent challenge for obtaining an oral therapeutic is an increased bioavailability of an orally administered compound.

Secondly, an oral administered protein compound must be absorbed during its progression through the gastrointestinal tract. The jejunum connects with the duodenum and the ileum and is specialized in absorption of digestion products such as sugars, amino acids and fatty acids, helped by the large surface area of the mucous membrane.

The epithelial cells lining the jejunum and ileum enables passive transport nutrients and active transport of amino acids, small peptides, vitamins, and glucose. Up-take of peptides and particular large protein molecules by the epithelia cells is thus highly critical for generating an oral pharmaceutical composition.

Multiple technologies are being explored and the present invention relates to use of fatty acid acylated amino acids which are previously known ingredients from cosmetic products and mainly used for increasing transdermal absorption.

Föger et al. described the impact of the molecular weight on oral absorption of hydrophilic peptide drugs and showed that the permeability decreased with increasing molecular weight of such hydrophilic peptide drugs (Amino Acids (2008) 25: 233-241, DOI 10.1007/s00726-007-0581-5).

The research into new surfactants with low irritant action has led to the development of different surfactants derived from amino acids (Mitjans et al., 2003; Benavides et al., 2004; Sánchez et al., 2006). FA-aa's are amino acid based surfactants and thus mild biodegradable surfactants with a low toxicity.

US2004147578 (WO 0207506) relates to fatty acid acylated amino acids used as permeation enhancers for hydrophobic molecules including hydrophobic macromolecules such as cyclosporine.

WO2001035998 relates to acylated amino acids used as transdermal and trans-mucosal absorption promoters for macromolecules, such as hydrophilic peptides or proteins.

WO2004064758 relates to an oral composition for delivering pharmaceutical peptides, such as insulin, growth hormone and GLP-1, wherein the active peptide is amidated at a site that is not naturally amidated. The compositions may comprise absorption enhancers, such acyl camitines exemplified using lauroylcamitine in formulations of PTH1-34.

US2005282756 is related to a dry powder composition comprising insulin and an absorption enhancer.

WO2003030865 is related to insulin compositions comprising surfactants such as ionic surfactants and does also contain oil or lipid compounds such as triglycerides and does further comprise long chain esterified fatty acids.

WO2004064758 is related to an oral pharmaceutical composition for delivering pharmaceutical peptides, comprising absorption enhancers such as acylcamitines, phospholipis and bile acids.

US2006093632 is related to injectable composition which forms a precipitate when injected into water resulting in a sustained release formulation,

EP0552405 disclose percutaneous absorption promoters for compounds such as indomethacin and derivatives of anthranilic acid.

The oral route of administration is highly complex for high molecular weight compounds, such as growth hormone and further improvements are needed to establishment an acceptable composition suitable for the treatment of patients, with an effective bioavailability of the growth hormone compound.

### SUMMARY

It has surprisingly been found that fatty acid amino acids (FA-aa's) e.g. N-acylated amino acids increase the absorption of growth hormone compounds after oral administration.

Due to their low toxicity and increasing effect on oral bioavailability of the therapeutic macromolecule, such as a hydrophilic peptide or protein, FA-aa's according to the present invention are valuable ingredients in oral pharmaceutical compositions. FA-aa's according to this invention are especially valuable in oral pharmaceutical compositions comprising growth hormone compounds. This is of interest for diseases that demand chronic administration of therapeutic macromolecules (e.g. growth hormone), but is not limited hereto, since the most non-invasive, non-toxic administration of drugs is generally favoured in any treatment, also for sporadic or bulk administration of therapeutics.

So far, no growth hormone compounds are available as oral formulation mainly due to the great challenges of enzymatic degradation and very low intestinal permeability of such compounds.

An aspect of the present invention is a pharmaceutical composition comprising certain amino acids acylated at their alpha-amino group with a fatty acid of 8 to 18 carbons and an active ingredient, such as a growth hormone compound. The composition of the pharmaceutical composition makes it suited for oral administration as an increased bioavailability is observed.

The present invention is related to pharmaceutical compositions, comprising FA-aa's acting as permeation enhancers suitable for oral administration of growth hormone compounds.

The invention may also solve further problems that will be apparent from the disclosure of the exemplary embodiments. The present invention is related to pharmaceutical compositions comprising FA-aa's suitable for increasing the absorption and/or bioavailability of growth hormone compounds, which is particularly important for a pharmaceutical composition to be administered orally.

As will be apparent from the disclosure herein, the compounds of interest are described as (fatty acid) acylated amino acids or fatty acid N-acylated amino acids or simply, for short, FA-aa's, describing compounds having an amino acid basic structure wherein the alpha amino group is acylated with a fatty acid, by formation of a peptide-like bond.

An aspect of the invention concerns a pharmaceutical composition comprising at least one growth hormone compound and at least one FA-aa. The FA-aa's described herein include a fatty acid moiety and an amino acid back-bone as the core structure. The compounds have been found to increase the bioavailability of growth hormone compounds when administered to the intestine. In a further aspect the composition comprise a FA-aa, wherein the fatty acid moiety has 12, 14 or 16 carbon atoms.

An increased stability of the growth hormone compound compared to human growth hormone is beneficial for obtaining a formulation that may be administered orally and/or with sufficient intervals. In a further aspect the composition comprises a growth hormone compound having a T½ above 30 minutes.

The invention in a further aspect relates to methods for increasing bioavailability of a growth hormone compound comprising the steps of including a FA-aa in a pharmaceutical composition of a growth hormone compound. Other methods of the invention aim at increasing the plasma concentration of a growth hormone compound and comprise the step of exposing the gastrointestinal tract of an individual to a pharmaceutical composition comprising a growth hormone compound and a FA-aa whereby an increased plasma concentration of said growth hormone compound is observed in said individual. It is also relevant to consider that including the FA-aa in the composition enables an increase in bioavailability or plasma concentration of the administered growth hormone compound that is larger than the increase observed when the compound is administered without including at least one FA-aa.

An aspect of the invention relates to a method for treatment of growth hormone relates diseases or disorders comprising administering a pharmaceutical composition comprising a growth hormone compound and at least one FA-aa.

### BRIEF DESCRIPTION OF DRAWINGS

FIG. 1. Mean plasma concentration of growth hormone analogue GH-A2 as function of time after intra-intestinal administration using a formulation with (solid markers) and without (open markers) C16-Glu analogous to the procedure described in Examples 8 and 11.

FIG. 2. The average AUC determined from the pharmacokinetic profile of compound GH-A1 formulated in aqueous buffers. The concentration of the compared fatty acid acylated amino acids is 30 mg/ml. The average AUC values are based on various numbers of in vivo studies: No enh. (69 studies, with n=6), C˜12-sarcosinate (2 studies, with n=6), and C16-sarcosinate (14 studies, with n=6).

FIG. 3. The average AUC determined from the pharmacokinetic profile of compoundGH-A2 formulated in aqueous buffers. The concentration of the compared fatty acid acylated amino acids is 30 mg/ml. The average AUC values are based on various numbers of in vivo studies: No enh. (12 studies, with n varying from 6-12), C10-Gly (1 study, n=12), C12-His (1 study, n=12), C12-Pro (1 study, n=12), C16-sarcosinate (5 studies, with varying n from 6-12) and C16-Glu (1 study, n=12).

FIG. 4. The average AUC determined from the pharmacokinetic profile of compound GH-A3 formulated in aqueous buffers. The concentration of the compared fatty acid acylated amino acids is 30 mg/ml. The average AUC values are based on various numbers of in vivo studies: No enh. (2 studies, with n=6), C8-His (2 studies, with n=6), C8-sarcosinate (2 studies, with n=6), C10-Asp (2 studies, with n=6), C10-Gln (2 studies, with n=6), C12-Trp (2 studies, with n=6), C12-sarcosinate (2 studies, with n=6), C12-Pro (2 studies, with n=6), C16-sarcosinate (7 studies, with n=6), C16-Glu (2 studies, with n=6) and C18=sarcosinate (2 studies, with n=6).

FIG. 5. The average AUC determined from the pharmacokinetic profile of compound GH-A1 formulated in aqueous buffers. The buffers are prepared with and without the fatty acid acylated amino acid C16-sarcosinate (30 mg/ml) and with or without Soy Bean Trypsin Inhibitor, SBTI (2%). The average AUC values are based on various numbers of in vivo studies: No enh. (69 studies, with n=6), C16-sarcosinate (14 studies, with n=6), SBTI (1 study, n=6), and C16-sarcosinate+SBTI (1 study, n=6).

FIG. 6. The average AUC determined from the pharmacokinetic profile of compound GH-A2 formulated in aqueous buffers. The buffers are prepared with and without the fatty acid acylated amino acid C16-sarcosinate (30 mg/ml) and with or without Soy Bean Trypsin Inhibitor, SBTI (2%). The average AUC values are based on various numbers of in vivo studies: No inh. (12 studies, with n varying from 6-12), C16-sarcosinate (5 studies, with varying n from 6-12), SBTI (3 studies, with varying n from 6-9), and C16-sarcosinate+SBTI (5 studies, with varying n from 6-9).

FIG. 7. The average AUC determined from the pharmacokinetic profile of compound GH-A3 formulated in aqueous buffers. The buffers are prepared with and without the fatty acid acylated amino acid C16-sarcosinate (30 mg/ml) and with or without Soy Bean Trypsin Inhibitor, SBTI (2%). The average AUC values are based on various numbers of in vivo studies: No inh. (2 studies, with n=6), C16-sarcosinate (7 studies, with n=6), SBTI (1 study, n=6), and C16-sarcosinate+SBTI (1 study, n=6).

### DESCRIPTION

### Selected Definitions

The term “polypeptide” and “peptide” as used herein means a compound composed of at least two amino acids connected by peptide bonds.

The term “amino acid” includes the group of the amino acids encoded by the genetic code which are herein referred to as standard amino acid. Further included are natural amino acids which are not encoded by the genetic code, as well as synthetic amino acids. Commonly known natural amino acids include γ-carboxyglutamate, hydroxyproline, omithine, sarcosine and phosphoserine. Commonly known synthetic amino acids comprise amino acids manufactured by chemical synthesis, such as Aib (alpha-aminoisobutyric acid), Abu (alpha-aminobutyric acid), Tie (tert-butylglycine), β-alanine, 3-aminomethyl benzoic acid, anthranilic acid.

The term “Protein” as used herein means a biochemical compound consisting of one or more polypeptides.

The term “drug”, “therapeutic”, “medicament” or “medicine” when used herein refer to an active ingredient used in a pharmaceutical composition, which may be used in therapy.

At least 300 amino acids have been described in nature but only twenty of these are typically found as components in human peptides and proteins.

Twenty standards amino acids are used by cells in peptide biosynthesis and these are specified by the general genetic code. The twenty standard amino acids are Alanine (Ala), Valine (Val), Leucine (Leu), Isoleucine (lile), Phenylalanine (Phe), Tryptophane (Trp), Methionine (Met), Proline (Pro), Apartic acid (Asp), Gltamic acid (Glu), Glycine (Gly), Serine (Ser), Threonine (Thr), Cysteine (Cys), Tyrosine (Tyr), Apsaragine (Asn), Glutamine (Gln), Lysine (Lys), Arginine (Arg) and Histidine (His). Sarcosine only differentiates from Glycine by an exchange of an —H with an —CH₃ and is for the following also considered an amino acid.

The standard amino acid may be divided into polar amino acid and non-polar amino acids. The polar amino acid are Aspartic acid (Asp), Glutamic acid (Glu), Lysine (Lys), Arginine (Arg), Histidine (His), Glutamine (Gln), Asparagine (Asn), Serine (Ser), Threonine (Thr) and Cysteine (Cys) and the non-polar amino acids are Glycine (Gly), Alanine (Ala), Leucine (Leu), Valine (Val), Isoleucine (lile), Methionine (Met), Proline (Pro), Tyrosine (Tyr), Tryptophane (Trp) and Phenylalanine (Phe).

The polar amino acid may be subdivided into non-charged polar amino acids (Glutamine (Gln), Asparagine (Asn), Serine (Ser), Threonine (Thr) and Cysteine (Cys)) and charged polar amino acids (Aspartic acid (Asp), Glutamic acid (Glu), Lysine (Lys), Arginine (Arg) and Histidine (His)).

The non-polar amino acids may be divided according to hydrophobicity, such that non-polar and hydrophobically neutral amino acids are Glycine and Alanine and the non-polar and hydrophobic amino acids are Leucine (Leu), Valine (Val), Isoleucine (lile), Methionine (Met), Proline (Pro), Tyrosine (Tyr), Tryptophane (Trp) and Phenylalanine (Phe).

The latter group may be dived based on the side chain being either aliphatic (Leucine (Leu), Valine (Val), Isoleucine (lile), Methionine (Met) and Proline (Pro)) or aromatic (Tyrosine (Tyr), Tryptophane (Trp), Phenylalanine (Phe)).

Fatty acid is a carboxylic acid with a long aliphatic tail (chain).

The terms “fatty acid amino acid”, “fatty acid N-acylated amino acid” or “acylated amino acid” or “fatty acid acylated amino acid” may be used interchangeable and refer when used herein to an amino acids that is acylated with a fatty acid at its alpha-amino group. The molecule is for short referred to as “FA-aa”.

With the term “oral bioavailability” is herein meant the fraction of the administered dose of drug that reaches the systemic circulation after having been administered orally. By definition, when a medication is administered intravenously, its bioavailability is 100%. However, when a drug is administered orally the bioavailability of the active ingredient decreases due to degradation, incomplete absorption and first-pass metabolism.

The term “biological activity” should represent the activity of a growth hormone compound in a relevant reference system, and may be measured in various assays known by a person skilled in the art as e.g. such as the BAF assay described in Example 1.

The term “surfactant” as used herein refers to any substance, in particular a detergent, that can adsorb at surfaces and interfaces, such as but not limited to liquid to air, liquid to liquid, liquid to container or liquid to any solid.

The term “non-ionic surfactant” as used herein refers to any substance, in particular a detergent, that can adsorb at surfaces and interfaces, like liquid to air, liquid to liquid, liquid to container or liquid to any solid and which has no charged groups in its hydrophilic group(s) (sometimes referred to as “heads”). The non-ionic surfactant may be selected from a detergent such as polysorbate, such as polysorbate-20, polysorbate-40, polysorbate-60, polysorbate-80, super refined polysorbates (where the term “super refined” is used by the supplier Croda for their high purity Tween products), poloxamers, such as poloxamer 188 and poloxamer 407, polyoxyethylene sorbitan fatty acid esters, polyoxyethylene derivatives such as alkylated and alkoxylated derivatives (Tweens, e.g. Tween-20 or Tween-80),

The term “co-surfactant” when used herein refers to an additional surfactant added to a composition or formulation, wherein a first surfactant is present.

The term “permeation enhancer” when used herein refers to biologicals or chemicals that promote the absorption of drugs.

The term “enhancer” is used when testing the ability of FA-aa's to increase the AUC of a given GH compound. The AUC of the GH compound is measured in the presence of test compound (enhancer) and compared to the AUC measured in the absence of enhancer (test compound).

The term “preservative” as used herein refers to a chemical compound which is added to a pharmaceutical composition to prevent or delay microbial activity (growth and metabolism). Examples of pharmaceutically acceptable preservatives are phenol, m-cresol and a mixture of phenol and m-cresol.

As used herein, the term “microemulsion pre-concentrate” means a composition, which spontaneously forms a microemulsion or a nanoemulsion, e.g., an oil-in-water microemulsion or nanoemulsion, swollen micelle, micellar solution, in an aqueous medium, e.g. in water or in the gastrointestinal fluids after oral application. The composition self-emulsifies upon dilution in an aqueous medium for example in a dilution of 1:5, 1:10, 1:50, 1:100 or higher. In one embodiment the composition according to the present invention forms the microemuslion or nanoemulsion comprising particles or domains of a size below 100 nm in diameter. The term “domain size” or “particle size” as used herein refers to repetitive scattering units and may be measured by e.g., small angle X-ray. Particle/droplet size may be based on dynamic light scattering (DLS), estimating the average diameter of dispersed particles or droplets. In one embodiment of the invention, the domain size is smaller than 150 nm, in another embodiment, smaller than 100 nm and in another embodiment, smaller than 50 nm, in another embodiment, smaller than 20 nm, in another embodiment, smaller than 15 nm, such as about 12 nm.

“SEDDS” (self emulsifying drug delivery systems) are herein defined as mixtures of a hydrophilic component, a surfactant, optionally a co-surfactant or lipid component and a therapeutic macromolecule that forms spontaneously a fine oil in water emulsion when exposed to aqueous media under conditions of gentle agitation or digestive motility that would be encountered in the GI tract. “SMEDDS” (self micro-emulsifying drug delivery systems) are herein defined as isotropic mixtures of a hydrophilic component a surfactant, optionally a co-surfactant or lipid component and a therapeutic macromolecule that rapidly form an oil in water microemulsion or nanoemulsion when exposed to aqueous media under conditions of gentle agitation or digestive motility that would be encountered in the GI tract.

“SNEDDS” (self nano-emulsifying drug delivery systems) are herein defined as isotropic mixtures of a hydrophilic component, at least one surfactant with HLB above 10, optionally a co-surfactant and optionally a lipid component and a therapeutic macromolecule that rapidly form a nanoemulsion (droplet size below 20 nm in diameter as e.g. measured by PCS or DLS) when exposed to aqueous media under conditions of gentle agitation or digestive motility that would be encountered in the GI tract.

As used herein, the term “emulsion” refers to a slightly opaque, opalescent or opaque colloidal coarse dispersion that is formed spontaneously or substantially spontaneously when its components are brought into contact with an aqueous medium.

As used herein, the term “microemulsion” refers to a clear or translucent, slightly opaque, opalescent, non-opaque or substantially non-opaque colloidal dispersion that is formed spontaneously or substantially spontaneously when its components are brought into contact with an aqueous medium.

A microemulsion is kinetically stable system (sometimes referred to as thermodynamically stable system) and contains homogenously dispersed particles or domains, for example of a solid or liquid state (e.g., liquid lipid particles or droplets), of a mean diameter of less than 150 nm as measured by standard light scattering techniques, e.g., using Wyatt DynaPro plate reader DLS or a MALVERN ZETASIZER Nano ZS. In one embodiment when the pharmaceutical composition according to the invention is brought into contact with an aqueous medium a microemulsion is formed which contains homogenously dispersed particles or domains of a mean diameter of less than 100 nm, such as less than 50 nm, less than 40 nm and less than 30 nm. Thus, the term “Z average (nm)” refers to the particle size of the particles or domains of said microemulsion. The term “PDI” is the abbreviation of the term “polydispersity index” and is a measure of the heterogeneity of sizes of molecules or particles in a mixture.

As used herein, the term “nanoemulsion” refers to a clear or translucent, slightly opaque, opalescent, non-opaque or substantially non-opaque colloidal dispersion with particle or droplet size below 20 nm in diameter (as e.g. measured by DLS or PCS) that is formed spontaneously or substantially spontaneously when its components are brought into contact with an aqueous medium. In one embodiment when the pharmaceutical composition according to the invention is brought into contact with an aqueous medium a microemulsion is formed which contains homogenously dispersed particles or domains of a mean diameter of less than 20 nm, such as less than 15 nm, less than 10 nm and greater than about 2-4 nm.

As used herein the term “spontaneously dispersible” when referring to a pre-concentrate refers to a composition that is capable of producing colloidal structures such as nanoemulsions, microemulsions, emulsions and other colloidal systems, when diluted with an aqueous medium when the components of the composition of the invention are brought into contact with an aqueous medium, e.g. by simple shaking by hand for a short period of time, for example for ten seconds. In one embodiment a spontaneously dispersible concentrate according to the invention is a SEDDS, SMEDDS or SNEDDS.

### Pharmaceutical Composition Comprising Fatty Acid Amino Acid

The present invention relates to a pharmaceutical composition comprising at least one growth hormone compound and at least one fatty acid amino acid (FA-aa). If not commercially available modified amino acids may easily be prepared by acylation of amino acids using acylation agents known in the art that react with the free alpha-amino group of the amino acid.

An aspect of the invention relates to a pharmaceutical composition comprising:
a) at least one growth hormone compound and
b) at least one fatty acid amino acid (FA-aa) or a salt thereof.

In one embodiment of the invention the composition is an oral pharmaceutical composition or a composition for oral administration comprising:
a) at least one growth hormone compound and
b) at least one fatty acid amino acid or a salt thereof.

In one embodiment of the invention, the pharmaceutical composition comprises at least one growth hormone compound and at least one FA-aa, based on a natural, a synthetic or a standard amino acid. The FA-aa consists of an amino acid residue and a fatty acid moiety or chain. The FA-aa is said to be based on an amino acid, when the FA-aa includes the same side chain as the amino acid which it is based on. Non-standard amino acids may include additional elements, such as a methyl instead of a hydrogen on the amino group, which may also be considered part of the structure when the FA-aa is based on a non-standard amino acid.

In one embodiment the amino acid residue of a FA-aa according to this invention is selected from the group of standard amino acids and sarcosine.

Without being bound by the theory the ability of an FA-aa to increase the bioavailability of a therapeutic protein may be linked to the ability of the FA-aa to increase the permeability of the cell layer of the intestine. An in vitro assay based on HT29-MTX (E12) cells may be used to determine if a given FA-aa increases the permeability of a cell layer for growth hormone compounds as described in Example 1 D and Example 20.

In one embodiment of the invention the pharmaceutical composition comprises a fatty acid amino acid that increases transportation of a growth hormone compound across HT29-MTX (E12) cells. In one embodiment the fatty acid amino acid increases transportation of a growth hormone compound by at least 1.5 fold, such as by at least 1.8 fold, such as by by 2 fold. In further embodiments the fatty acid amino acid comprised by the composition of the invention increases transportation of a growth hormone compound by at least 3, 4 or 5 fold.

In one embodiment the amino acid residue of a FA-aa according to this invention is selected from the group comprising Alanine, Glycine, Proline, Isoleucine, Valine, Methionine, Tyrosine, Tryptophan, Asparagine, Glutamine, Aspartic acid, Glutamic acid, Lysine, Arginine, Histidine, Serine, Threonine, Leucine and Phenylalanine.

In one embodiment of the invention, the pharmaceutical composition comprises at least one growth hormone compound and at least one FA-aa, wherein the FA-aa is based on a polar or non-polar amino acid.

In one embodiment of the invention, the pharmaceutical composition comprises at least one growth hormone compound and at least one FA-aa, wherein the FA-aa is based on a nonpolar amino acid selected from the group consisting of: Glycine (Gly), Alanine (Ala), Leucine (Leu), Valine (Val), Isoleucine (lile), Methionine (Met), Proline (Pro), Tyrosine (Tyr), Tryptophane (Trp), Phenylalanine (Phe) and Sarcosine.

In one embodiment of the invention the non-polar amino acid is selected from the group of non-polar hydrophobically neutral amino acids consisting of Glycine (Gly), Alanine (Ala) and Sarcosine (Sarc).

In one embodiment of the invention the non-polar amino acid is selected from the group of non-polar hydrophobic amino acids consisting of: Leucine (Leu), Valine (Val), Isoleucine (lile), Methionine (Met), Proline (Pro), Tyrosine (Tyr), Tryptophane (Trp) and Phenylalanine (Phe).

In one embodiment of the invention the non-polar amino acid is selected from the group of non-polar hydrophobic aliphatic amino acids consisting of: Leucine (Leu), Valine (Val), Isoleucine (lile), Methionine (Met) and Proline (Pro),

In one embodiment of the invention the non-polar amino acid is selected from the group of non-polar hydrophobic aromatic amino acids consisting of: Tyrosine (Tyr), Tryptophane (Trp) and Phenylalanine (Phe).

In one embodiment of the invention, the pharmaceutical composition comprises at least one growth hormone compound and at least one FA-aa, wherein the FA-aa is based on a polar amino acid selected from the group consisting of: Aspartic acid (Asp), Glutamic acid (Glu), Lysine (Lys), Arginine (Arg), Histidine (His), Glutamine (Gln), Asparagine (Asn), Serine (Ser), Threonine (Thr) and Cysteine (Cys).

In one embodiment of the invention, the pharmaceutical composition comprises at least one growth hormone compound and at least one FA-aa, wherein the FA-aa is based on a charged polar amino acid selected from the group consisting of: Aspartic acid (Asp), Glutamic acid (Glu), Lysine (Lys), Arginine (Arg) and Histidine (His).

In one embodiment the FA′aa is based on a charged polar amino acid selected from the group consisting of: Lysine (Lys), Arginine (Arg) and Histidine (His). In one embodiment the FA′aa is based on a charged polar amino acid selected from the group consisting of: Aspartic acid (Asp) and Glutamic acid (Glu),

In one embodiment of the invention, the pharmaceutical composition comprises at least one growth hormone compound and at least one FA-aa, wherein the FA-aa is based on a non-charged polar amino acid selected from the group consisting of: Glutamine (Gln), Asparagine (Asn), Serine (Ser), Threonine (Thr) and Cysteine (Cys).

In one embodiment the FA-aa is based on a non-charged polar amino acid selected from the group consisting of: Glutamine (Gln) and Asparagine (Asn).

In one embodiment the FA-aa is based on a non-charged polar amino acid selected from the group consisting of: Serine (Ser), Threonine (Thr) and Cysteine (Cys).

In one embodiment the FA-aa is based on a non-charged polar amino acid selected from the group consisting of: Glutamine (Gln), Asparagine (Asn), Serine (Ser) and Threonine (Thr).

In one further embodiment the FA-aa is based on an amino acid selected from the group of amino acids consisting of Aspartic acid (Asp), Glutamic acid (Glu), Sarcosine (Sarc), Glycine (Gly), Lysine (Lys), Arginine (Arg), Histidine (His), Glutamine (Gln), Asparagine (Asn), Serine (Ser), Threonine (Thr), Tyrosine (Tyr) and Cysteine (Cys).

In one embodiment the FA-aa is based on an amino acid selected from the group consisting of Aspartic acid (Asp), Glutamic acid (Glu), Sarcosine (Sarc), Glycine (Gly), Glutamine (Gln), Asparagine (Asn), Serine (Ser), Threonine (Thr), Tyrosine (Tyr) and Cysteine (Cys).

In one embodiment the FA-aa is based on an amino acid selected from the group consisting of Aspartic acid (Asp), Glutamic acid (Glu), Sarcosine (Sarc), Glycine (Gly), Glutamine (Gln), Asparagine (Asn), Serine (Ser) and Threonine (Thr).

In one embodiment the FA-aa is based on an amino acid selected from the group consisting of Aspartic acid (Asp), Asparagine (Asn), Glutamic acid (Glu), Sarcosine (Sarc) and Glutamine (Gln).

In one embodiment the FA-aa is based on an amino acid selected from the group consisting of Aspartic acid (Asp), Glutamine (Gln), Glutamic acid (Glu) and Sarcosine (Sarc).

In one embodiment the FA-aa is based on an amino acid selected from the group consisting of Glutamine (Gln), Glutamic acid (Glu) and Sarcosine (Sarc).

According to this invention the FA-aa comprises an amino residue and a fatty acid attached to the amino acid by acylation of said amino acid's alpha-amino group, which gives rise to a —C(═O)— group neighbouring the amino acid's alpha-amino group. The carbonyl and the aliphatic chain originating from the fatty acid are collectively referred to as the fatty acid chain or the fatty acid moiety.

In one embodiment the carboxyl group of the amino acid residue may be in the form of a carboxylic acid (—COOH) or in the form of a carboxylate ion (COO⁻). The carboxylate ion usually forms a salt and the carboxyl-group of the amino acid residue in such complexes is usually denoted “-ate”, such as histidinate, tryptophanate and sarcosinate. The —COOH form is referred to as the form of its free acid, whereas the carboxylate ion in complex with a monovalent cation is in the form of a salt.

In one embodiment, the amino acid residue according to this invention is of the form of its free acid or a salt thereof.

In one embodiment the amino acid residue according to this invention is in the form of a sodium (Na⁺) salt or potassium (K⁺) salt.

In one embodiment the FA-aa according to this invention comprises a fatty acid moiety of 8 to 18 carbon atoms.

In one embodiment the FA-aa according to this invention comprises a fatty acid moiety of 10 to 18 carbon atoms.

In one embodiment the FA-aa according to this invention comprises a fatty acid moiety of 12 to 18 carbon atoms.

In one embodiment the FA-aa according to this invention comprises a fatty acid moiety of 12 to 16 carbon atoms.

In one embodiment the FA-aa according to this invention comprises a fatty acid moiety of 14 to 18 carbon atoms.

In one embodiment the FA-aa according to this invention comprises a fatty acid moiety of 14 to 16 carbon atoms.

In one embodiment a FA-aa according to this invention comprises a fatty acid moiety of 10 carbon atoms.

In one embodiment a FA-aa according to this invention comprises a fatty acid moiety of 12 carbon atoms.

In one embodiment a FA-aa according to this invention comprises a fatty acid moiety of 14 carbon atoms.

In one embodiment a FA-aa according to this invention comprises a fatty acid moiety of 16 carbon atoms.

In one embodiment a FA-aa according to this invention comprises a fatty acid moiety of 18 carbon atoms.

In one embodiment the fatty acid moiety may be either a saturated or unsaturated fatty acid moiety.

In one embodiment a FA-aa according to this invention the fatty acid moiety is located at the alpha amino group of the amino acid.

In one embodiment a FA-aa according to this invention comprises an amino acid residue in the form of its free acid or a as a salt thereof. A FA-aa according to the present invention may be represented by the general formula;

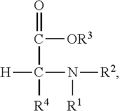

wherein R1 is a fatty acid chain comprising from 8 to 18 carbons, R2 is either H (i.e. hydrogen) or CH₃ (i.e. methyl group), R3 is either H, or absent, and R4 is an amino acid side chain of a polar or non-polar amino acid as described above.

In one embodiment a FA-aa according to the present invention may be represented by the general formula:

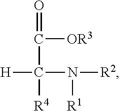

wherein R1 is a fatty acid chain comprising 8 to 18 carbon atoms, R2 is either H (i.e. hydrogen) or CH₃ (i.e. methyl group), R3 is either H, or absent, and R4 is an amino acid side chain of a polar or non-polar amino acid as described above.

In one embodiment a FA-aa according to the present invention may be represented by the general formula:

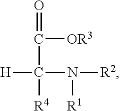

wherein

### R1 is —C(═O)—(CH₂)₆₋₁₆—CH₃,

R2 is H (i.e. hydrogen) or CH3 (i.e. methyl group) or a valence bond when R2 is covalently attached to R4, and
R3 is either H, or absent, and
R4 is an amino acid side chain also including —(CH₂)₃— when covalently attached to R2 as in Proline.

In one embodiment the carboxyl group of the amino acid residue may be in the form of a carboxylic acid or in the form of a carboxylate ion this is reflected in the structure where R3 is either H (carboxylic acid), or absent, which describes the carboxylate ion. This ion will usually be in complex with a cat-ion such as Na⁺ e.g. as a salt (referred to as a salt thereof in the broadest definition. The carboxyl group of the amino acid residue in such complexes is de-protonated (COO⁻) usually denoted “-ate”, such as histidinate, tryptophanate and sarcosinate.

The fatty acid amino acid (FA-aa) as described herein above thus defines an amino acid like structure wherein, R1 is obtained by linkage of a fatty acid via an amide/peptide bond to the amino group of the amino acid. This results in formation of the —C(═O)—(CH₂)₆₋₁₆—CH₃ structure when the starting fatty acid is a saturated fatty acid comprising 8-18 carbon atoms. In alternative embodiments, where the starting fatty acid is an unsaturated fatty acid the specific structure will be different.

The fatty acid amino acid further comprises an amino acid side chain referred to as R4 in the formula above. Of the natural amino acid, proline has special structure, as the side chain is covalently linked to the amino group. In the structure above the proline side chain is defined by R2 being a valence bond covalently attached to R4, when R4 is —(CH₂)₃.

In one embodiment a FA-aa according to this invention may be chosen from the group consisting of formula (a), (b), (c), (d), (e), (f), (g), (h), (i), (j), (k), (l), (m), (n), (o), (p), (q) (r), (s) or (t) shown below, wherein R1 is an aliphatic chain comprising 7 to 17 carbons, R2 is either H (i.e. hydrogen) or CH₃ (i.e. methyl group), and R3 is either H, or absent.

In one embodiment a FA-aa according to this invention may be chosen from the group consisting of formula (a), (b), (c), (d), (e), (f), (g), (h), (i), (j), (k), (l), (m), (n), (o), (p), (q) (r), (s) or (t) shown below, wherein R1 is an aliphatic chain comprising 9 to 15 carbon atoms, R2 is either H (i.e. hydrogen) or CH₃ (i.e. methyl group), and R3 is either H, or absent.

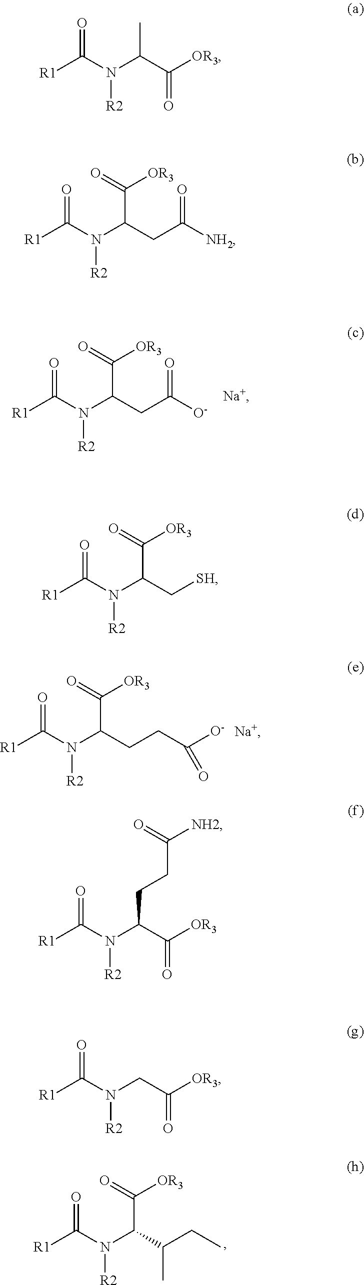

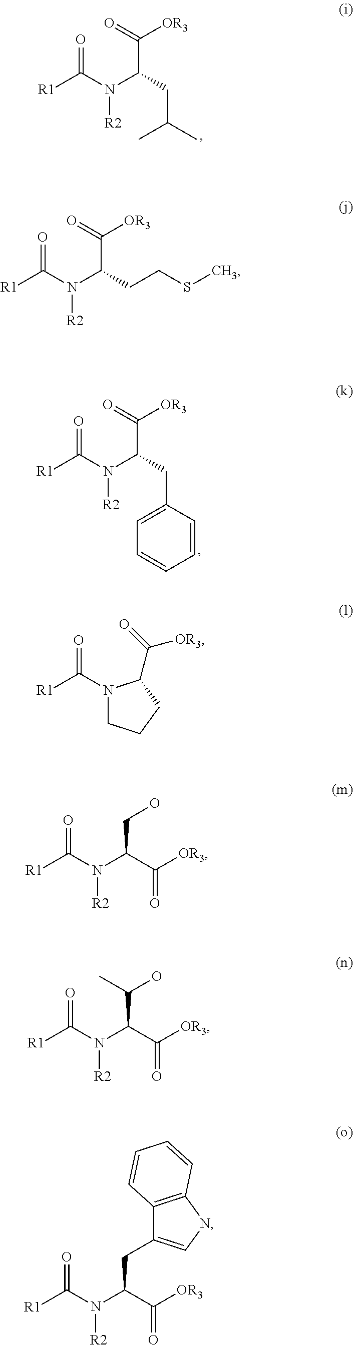

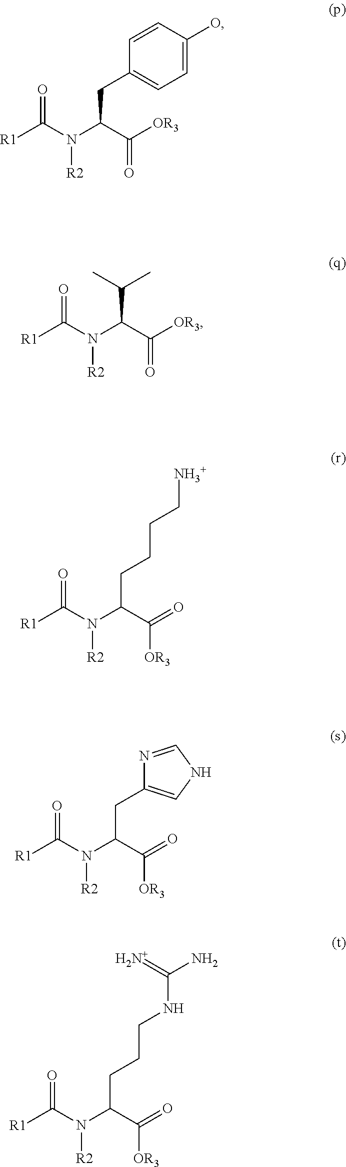

In contrast to general formula above, the carbonyl group (—C(═O)) is part of the structure and thus not part of R1

In addition to the naturally amino acid side chain, the FA-aa according to the invention may be derived from amino acid variants such as sarcosine. Referring to the formula above sarcosine may be viewed as a Glycine amino acid residue with an additional methyl group covalently bound to the nitrogen atom e.g. R2=CH3 and R4=H and the amino acid residue of the FA-aa so defined equals the structure of sacosine included herein as an amino acid.

In one embodiments the FA-aa can be selected from the group of FA-aa including 8 carbon atoms in the fatty acid moiety consisting of: Sodium caprylic alaninate, N-octanoyl-L-alanine, Sodium caprylic isoleucinate, N-octanoyl-L-isoleucine, Sodium caprylic leucinate, N-octanoyl-L-leucine, Sodium caprylic methioninate, N-octanoyl-L-methionine, Sodium caprylic phenylalaninate, N-octanoyl-L-phenylalanine, Sodium caprylic prolinate, N-octanoyl-L-proline, Sodium caprylic threoninate, N-octanoyl-L-threonine, Sodium caprylic serinate, N-octanoyl-L-serine, Sodium caprylic tryptophanate, N-octanoyl-L-tryptophane, Sodium caprylic valinate, N-octanoyl-L-valine, Sodium caprylic sarcosinate and N-octanoyl-L-sarcosine, Sodium caprylic tyrosinate, N-octanoyl-L-tyrosine, Sodium caprylic Glycinate, N-octanoyl-L-glycine Sodium caprylic glutaminate, N-octanoyl-L-glutamine, Sodium caprylic asparaginate, N-octanoyl-L-asparagine Sodium caprylic aspartic acid, N-octanoyl-L-aspartic acid, Sodium caprylic glutamic acid, N-octanoyl-L-glutamic acid, Sodium caprylic Cysteinate, N-octanoyl-L-cysteine, Sodium caprylic Lysinate, N-octanoyl-L-Lysine, Sodium caprylic arginate, N-octanoyl-L-arginine, Sodium caprylic histinate, N-octanoyl-L-histidine

In one embodiment the FA-aa can be selected from the group of FA-aa's including carbon atom in the fatty acid moiety consisting of: Sodium capric alaninate, N-decanoyl-L-alanine, Sodium capric isoleucinate, N-decanoyl-L-isoleucine, Sodium capric leucinate, N-decanoyl-L-leucine, Sodium capric methioninate, N-decanoyl-L-methionine, Sodium capric phenylalaninate, N-decanoyl-L-phenylalanine, Sodium capric prolinate, N-decanoyl-L-proline, Sodium capric threoninate, N-decanoyl-L-threonine, Sodium capric serinate, N-decanoyl-L-serine, Sodium capric tryptophanate, N-decanoyl-L-tryptophane, Sodium capric valinate, N-decanoyl-L-valine, Sodium capric sarcosinate and N-decanoyl-L-sarcosine, Sodium capric tyrosinate, N-decanoyl-L-tyrosine, Sodium capric Glycinate, N-decanoyl-L-glycine Sodium capric glutaminate, N-decanoyl-L-glutamine, Sodium capric asparaginate, N-decanoyl-L-asparagine Sodium capric aspartic acid, N-decanoyl-L-aspartic acid, Sodium capric glutamic acid, N-decanoyl-L-glutamic acid, Sodium capric Cysteinate, N-decanoyl-L-cysteine, Sodium capric Lysinate, N-decanoyl-L-Lysine, Sodium capric arginate, N-decanoyl-L-arginine, Sodium capric histinate, N-decanoyl-L-histidine

In one embodiment the FA-aa can be selected from the group of FA-aa's including 12 carbon atom in the fatty acid moiety consisting of: Sodium lauroyl alaninate, N-dodecanoyl-L-alanine, Sodium lauroyl isoleucinate, N-dodecanoyl-L-isoleucine, Sodium lauroyl leucinate, N-dodecanoyl-L-leucine, Sodium lauroyl methioninate, N-dodecanoyl-L-methionine, Sodium lauroyl phenylalaninate, N-dodecanoyl-L-phenylalanine, Sodium lauroyl prolinate, N-dodecanoyl-L-proline, Sodium lauroyl threoninate, N-dodecanoyl-L-threonine, Sodium lauroyl serinate, N-dodecanoyl-L-serine, Sodium lauroyl tryptophanate, N-dodecanoyl-L-tryptophane, Sodium lauroyl valinate, N-dodecanoyl-L-valine, Sodium lauroyl sarcosinate and N-dodecanoyl-L-sarcosine, Sodium lauroyl tyrosinate, N-dodecanoyl-L-tyrosine, Sodium lauroyl Glycinate, N-dodecanoyl-L-glycine Sodium lauroyl glutaminate, N-dodecanoyl-L-glutamine, Sodium lauroyl asparaginate, N-dodecanoyl-L-asparagine Sodium lauroyl aspartic acid, N-dodecanoyl-L-aspartic acid, Sodium lauroyl glutamic acid, N-dodecanoyl-L-glutamic acid, Sodium lauroyl Cysteinate, N-dodecanoyl-L-cysteine, Sodium lauroyl Lysinate, N-dodecanoyl-L-Lysine, Sodium lauroyl arginate, N-dodecanoyl-L-arginine, Sodium lauroyl histinate, N-dodecanoyl-L-histidine

In one embodiment the FA-aa can be selected from the group of FA-aa's including 14 carbon atoms in the fatty acid moiety consisting of: Sodium myristoyl alaninate, N-tetradecanoyl-L-alanine, Sodium myristoyl isoleucinate, N-tetradecanoyl-L-isoleucine, Sodium myristoyl leucinate, N-tetradecanoyl-L-leucine, Sodium myristoyl methioninate, N-tetradecanoyl-L-methionine, Sodium myristoyl phenylalaninate, N-tetradecanoyl-L-phenylalanine, Sodium myristoyl prolinate, N-tetradecanoyl-L-proline, Sodium myristoyl threoninate, N-tetradecanoyl-L-threonine, Sodium myristoyl serinate, N-tetradecanoyl-L-serine, Sodium myristoyl tryptophanate, N-tetradecanoyl-L-tryptophane, Sodium myristoyl valinate, N-tetradecanoyl-L-valine, Sodium myristoyl sarcosinate and N-tetradecanoyl-L-sarcosine, Sodium myristoyl tyrosinate, N-tetradecanoyl-L-tyrosine, Sodium myristoyl Glycinate, N-tetradecanoyl-L-glycine Sodium myristoyl glutaminate, N-tetradecanoyl-L-glutamine, Sodium myristoyl asparaginate, N-tetradecanoyl-L-asparagine Sodium myristoyl aspartic acid, N-tetradecanoyl-L-aspartic acid, Sodium myristoyl glutamic acid, N-tetradecanoyl-L-glutamic acid, Sodium myristoyl Cysteinate, N-tetradecanoyl-L-cysteine, Sodium myristoyl Lysinate, N-tetradecanoyl-L-Lysine, Sodium myristoyl arginate, N-tetradecanoyl-L-arginine, Sodium myristoyl histinate, N-tetradecanoyl-L-histidine

In one embodiment a FA-aa can be selected from the group of FA-aa's including 16 carbon atoms in the fatty acid moiety consisting of: Sodium palmitoyl alaninate, N-hexadecanoyl-L-alanine, Sodium palmitoyl isoleucinate, N-hexadecanoyl-L-isoleucine, Sodium palmitoyl leucinate, N-hexadecanoyl-L-leucine, Sodium palmitoyl methioninate, N-hexadecanoyl-L-methionine, Sodium palmitoyl phenylalaninate, N-hexadecanoyl-L-phenylalanine, Sodium palmitoyl prolinate, N-hexadecanoyl-L-proline, Sodium palmitoyl threoninate, N-hexadecanoyl-L-threonine, Sodium palmitoyl serinate, N-hexadecanoyl-L-serine, Sodium palmitoyl tryptophanate, N-hexadecanoyl-L-tryptophane, Sodium palmitoyl valinate, N-hexadecanoyl-L-valine, Sodium palmitoyl sarcosinate and N-hexadecanoyl-L-sarcosine, Sodium palmitoyl tyrosinate, N-hexadecanoyl-L-tyrosine, Sodium palmitoyl Glycinate, N-hexadecanoyl-L-glycine Sodium palmitoyl glutaminate, N-hexadecanoyl-L-glutamine, Sodium palmitoyl asparaginate, N-hexadecanoyl-L-asparagine Sodium palmitoyl aspartic acid, N-hexadecanoyl-L-aspartic acid, Sodium palmitoyl glutamic acid, N-hexadecanoyl-L-glutamic acid, Sodium palmitoyl Cysteinate, N-hexadecanoyl-L-cysteine, Sodium palmitoyl Lysinate, N-hexadecanoyl-L-Lysine, Sodium palmitoyl arginate, N-hexadecanoyl-L-arginine, Sodium palmitoyl histinate, N-hexadecanoyl-L-histidine

In one embodiment a FA-aa can be selected from the group of FA-aa's including 18 carbon atoms, wherein the fatty acid moiety is derived from Stearic acid. The FA′aa may thus be selected from the group consisting of: Sodium stearoyl alaninate, N-octadecanoyl-L-alanine, Sodium stearoyl isoleucinate, N-octadecanoyl-L-isoleucine, Sodium stearoyl leucinate, N-octadecanoyl-L-leucine, Sodium stearoyl methioninate, N-octadecanoyl-L-methionine, Sodium stearoyl phenylalaninate, N-octadecanoyl-L-phenylalanine, Sodium stearoyl prolinate, N-octadecanoyl-L-proline, Sodium stearoyl threoninate, N-octadecanoyl-L-threonine, Sodium stearoyl serinate, N-octadecanoyl-L-serine, Sodium stearoyl tryptophanate, N-octadecanoyl-L-tryptophane, Sodium stearoyl valinate, N-octadecanoyl-L-valine, Sodium stearoyl sarcosinate and N-octadecanoyl-L-sarcosine, Sodium stearoyl tyrosinate, N-octadecanoyl-L-tyrosine, Sodium stearoyl Glycinate, N-octadecanoyl-L-glycine Sodium stearoyl glutaminate, N-octadecanoyl-L-glutamine, Sodium stearoyl asparaginate, N-octadecanoyl-L-asparagine Sodium stearoyl aspartic acid, N-octadecanoyl-L-aspartic acid, Sodium stearoyl glutamic acid, N-octadecanoyl-L-glutamic acid, Sodium stearoyl Cysteinate, N-octadecanoyl-L-cysteine, Sodium stearoyl Lysinate, N-octadecanoyl-L-Lysine, Sodium stearoyl arginate, N-octadecanoyl-L-arginine, Sodium stearoyl histinate, N-octadecanoyl-L-histidine.

In one embodiment a FA-aa can be selected from the group of FA-aa's including 18 carbon atoms, wherein the fatty acid moiety is derived from oieic acid. The FA′aa may thus be selected from the group consisting of: Sodium oleoyl alaninate, N-(E)-octadec-9-enoyl-L-alanine, Sodium oleoyl isoieucinate, N-(E)-octadec-9-enoyl-L-isoleucine, Sodium oleoyl leucinate, N-(E)-octadec-9-enoyl-L-leucine, Sodium oeoyl methioninate, N-(E)-octadec-9-enoyl-L-methionine, Sodium oleoyl phenylalaninate, N-(E)-octadec-9-enoyl-L-phenylalanine, Sodium oleoyl prolinate, N-(E)-octadec-9-enoyl-L-proline, Sodium oleoyl threoninate, N-(E)-octadec-9-enoyl-L-threonine, Sodium oleoyl serinate, N-(E)-octadec-9-enoyl-L-serine, Sodium oleoyl tryptophanate, N-(E)-octadec-9-enoyl-L-tryptophane, Sodium oleoyl valinate, N-(E)-octadec-9-enoyl-L-valine, Sodium oleoyl sarcosinate and N-(E)-octadec-9-enoyl-L-sarcosine, Sodium oleoyl tyrosinate, N-(E)-octadec-9-enoyl-L-tyrosine, Sodium oleoyl Glycinate, N-(E)-octadec-9-enoyl-L-glycine Sodium oleoyl glutaminate, N-(E)-octadec-9-enoyl-L-glutamine, Sodium oleoyl asparaginate, N-(E)-octadec-9-enoyl-L-asparagine Sodium oleoyl aspartic acid, N-(E)-octadec-9-enoyl-L-aspartic acid, Sodium oleoyl glutamic acid, N-(E)-octadec-9-enoyl-L-glutamic acid, Sodium oleoyl Cysteinate, N-(E)-octadec-9-enoyl-L-cysteine, Sodium oleoyl Lysinate, N-(E)-octadec-9-enoyl-L-Lysine, Sodium oleoyl arginate, N-(E)-octadec-9-enoyl-L-arginine, Sodium oleoyl histinate, N-(E)-octadec-9-enoyl-L-histidine.

In one embodiment, wherein the FA-aa has 12 carbon atoms in the fatty acid moiety the FAaa is based on an amino acids selected from the group consisting of: Glutamine (Gln), Glutamic acid (Glu), Aspartic acid (Asp), Sarcosine (Sarc), Leucine (Leu), Valine (Val), Tyrosine (Tyr) and Tryptophan (Trp).

In one embodiment, wherein the FA-aa has 14 carbon atoms in the fatty acid moiety the FAaa is based on an amino acids selected from the group consisting of: Glutamine (Gin), Glutamic acid (Glu), D-Glutamic acid (D-Glu), Leucine (Leu), Valine (Val) and Sarcosine (Sarc).

In one embodiment, wherein the FA-aa has 16 carbon atoms in the fatty acid moiety the FAaa is based on an amino acids selected from the group consisting of: Proline (Pro), Glutamine (Gin), Glutamic acid (Glu), D-Glutamic acid (D-Glu), Aspartic acid (Asp), D-Aspartic acid (D-Asp) and Sarcosine (Sarc).

In one embodiment, wherein the FA-aa has 16 carbon atoms in the fatty acid moiety the FA-aa is based on an amino acid selected from the group consisting of: Aspartic acid (Asp), Glutamic acid (Glu) and Sarcosine (Sarc).

In one embodiment, wherein the FA-aa has 16 carbon atoms in the fatty acid moiety the FAaa is based on an amino acids selected from the group consisting of: Glutamine (Gln), Glutamic acid (Glu) and Sarcosine (Sarc).

In one embodiment an amino acid residue of a FA-aa according to this invention is an amino acid that is not encoded by the genetic code.

In one embodiment an amino acid residue of a FA-aa according to this invention is Sarcosine.

In one embodiment an amino acid residue of a FA-aa according to this invention is a free acid or salt form of an amino acid that is not encoded by the genetic code.

In one embodiment an amino acid residue of a FA-aa according to this invention is the free acid Sarcosine or the salt in the form of Sarcosinate.

In one embodiment of the invention the oral pharmaceutical composition comprises of at least one growth hormone compound and at least on FA-aa.

### Growth Hormone Compound

With the term “growth hormone compound” as used herein, is meant a growth hormone molecule retaining at least some of the functionalities of human growth hormone identified by SEQ ID NO 1 and the overall structure hereof including the two intra-molecular di-sulfphide bonds connecting C53 with C165 and C182 with C189 or corresponding amino acid residues in growth hormone variants. The structure of growth hormone proteins is composed of four helixes (helix 1-4) connected by three loops (L1-3), and a C-terminal segment. In human growth hormone helix 1 is defined by AA residue 6-35, helix 2 is defined by AA residues 71-98, helix 3 is defined by AA residue 107-127 and helix for is defined as AA residues 155-184.

A BAF assay is frequently used to determine the biological activity of a growth hormone protein or compound (see below).

The terms “growth hormone variant” and “growth hormone analogue” as used herein means a growth hormone protein which has an amino acid sequence which is derived from the structure of a naturally occurring growth hormone, for example that of human growth hormone identified by SEQ ID NO 1. The term is thus used for a growth hormone protein wherein one or more amino acid residues of the growth hormone sequence has/have been substituted by other amino acid residue(s) and/or wherein one or more amino acid residue(s) have been deleted from the growth hormone and/or wherein one or more amino acid residues have been added and/or inserted to the growth hormone.

The term “growth hormone derivative” as used herein refers to a chemically modified growth hormone protein or analogue thereof, wherein the modification(s) are in the form of attachment of amides, carbohydrates, alkyl groups, esters, PEGylations, and the like.

A growth hormone derivative according to the invention is a naturally occurring growth hormone or an growth hormone analogue which has been modified, e.g. by chemically introducing a side chain in one or more positions of the growth hormone backbone or by oxidizing or reducing groups of the amino acid residues in the growth hormone or by converting a free carboxylic group to an ester group or to an amide group. Other derivatives are obtained by acylating a free thiol group introduced via single amino acid mutations in the growth hormone sequence. Herein, the term “acylated growth hormone” covers modification of growth hormone by attachment of one or more lipophilic substituents optionally via a linker to the growth hormone protein.

A “lipophilic substituent” is herein understood as a side chain consisting of a fatty acid or a fatty diacid attached to the growth hormone protein or analogue, optionally via a linker.

A growth hormone derivative is thus human growth hormone or a human growth hormone analogue which comprises at least one covalent modification attached to one or more amino acids, such as to one or more amino acid side chains of the growth hormone or growth hormone variant or analogue.

The term “growth hormone fusion” is used herein to protein molecules that include a growth hormone sequence linked to a second protein sequence by means of a peptide bond, which is usually obtained by expression of the fusion protein using a recombinant expression vector linking a DNA sequence encoding said growth hormone sequence with a DNA sequence encoding said second protein optionally including a linker sequence. Growth hormone fusions include, but not limited to, fusions comprising an antibody Fc region or regions and/or an albumin protein.

The term “growth hormone compound” as used herein collectively refers to a growth hormone molecule retaining substantially the functional characteristics of human growth hormone. The compound may thus be a growth hormone, a growth hormone fusion protein, a growth hormone variant or analogue or a growth hormone derivative including also acylated growth hormone.

In one embodiment a growth hormone analogue according to the invention comprises less than 8 modifications (substitutions, deletions, additions) relative to human growth hormone.

In one embodiment a growth hormone analogue comprises less than 7 modifications (substitutions, deletions, additions) relative to human growth hormone. In one embodiment a growth hormone analogue comprises less than 6 modifications (substitutions, deletions, additions) relative to human growth hormone.

In one embodiment a growth hormone analogue comprises less than 5 modifications (substitutions, deletions, additions) relative to human growth hormone. In one embodiment a growth hormone analogue comprises less than 4 modifications (substitutions, deletions, additions) relative to human growth hormone. In one embodiment a growth hormone analogue comprises less than 3 modifications (substitutions, deletions, additions) relative to human growth hormone. In one embodiment a growth hormone analogue comprises less than 2 modifications (substitutions, deletions, additions) relative to human growth hormone.

In a series of embodiment the growth hormone analogue of the growth hormone is at least 95, 96, 97, 98 or 99% identical to human growth hormone identified by SEQ ID NO: 1.

The growth hormone compound preferably has increase plasma T₁₂ compared to wt human growth hormone. That may be provided by various means known to the person skilled in the art, such as point mutations stabilizing the protein from degradation. The circulation time of a growth hormone compound may also be obtained by linkage covalently or non-covalently to serum proteins. Serum albumin may be used by direct conjugation (optionally including a linker) or by protein fusion with a growth hormone or variant thereof. Alternatively chemical linkage to albumin may also be considered as well as fusion or linkage with antibody Fc regions. Non-covalent attachment to albumin may be obtained through the use of albumin binders such as acyl groups covalently bond to growth hormone.

In one embodiment the growth hormone compound has an increased T ½ compared to human growth hormone, wherein the T ½ is measured after intravenous (i.v.) or subcutaneous (s.c.) administration to rats as described in Example 1.C. It is noted that wt human growth hormone has a T ½ of approximately 12-14 minutes in the described assay. In on embodiment the growth hormone compound has a T ½ above 30 minutes. In further embodiments the T ½ is above 60 minutes or 1 hour, such as above 2 hours or preferably above 4 hours.

In one embodiment a growth hormone compound is a growth hormone derivative that is a growth hormone or variant thereof that is acylated in one or more amino acids of the growth hormone protein.

In one embodiment a growth hormone compound is a growth hormone variant that is stabilized towards proteolytic degradation (by specific mutations), and such variants may further be acylated in one or more amino acids of the growth hormone protein.

Non-limiting examples of growth hormone proteins that are stabilized towards proteolytic degradation (by specific mutations) may e.g. be found in WO2010/084173 and WO 2011/089250.

It is further noted and also described below that stabilization of the growth hormone compound may be obtained by including a protease inhibitor in the formulation.

Protease-stabilized growth hormone protein variants include variants where an additional disulfide bridge is introduced. The additional di-sulfide bridge preferably connects L3 with helix 2. This may be obtained by introducing two extra Cys aa residues, which in preferred embodiments are substituted for the wt aa residue in positions corresponding to AA84 or AA85 in H2 and AA143 or AA144 in L3 of SEQ ID No. 1. The growth hormone variant may thus according to the invention preferably comprise a pair of mutations corresponding to L73C/S132C, L73C/F139C, R77C/I1138C, R77C/F139C, L81C/Q141C, L81C/Y143C, Q84C/Y143C, Q84C/S144C, S85C/Y143C, S85C/S144C, P89C/F146C, F92C/F146C or F92C/T148C in SEQ ID No.1. In a further embodiment the growth hormone variant comprises a pair of mutations corresponding to L81C/Y143C, Q84C/Y143C, S85C/Y143C, S85C/S144C or F92C/T148C in SEQ ID No. 1.

In one embodiment the growth hormone compound is a growth hormone derivative, such as a mono-substituted compound having only one acylation group attached to an internal amino acid residue in a growth hormone protein or a variant thereof such as the protease stabilized growth hormone variants described above.

A non-limiting list of acylated growth hormone compounds suitable for the pharmaceutical composition of the invention may e.g. be found in WO2011/089255.

Examplatory compounds including T ½ as measured are listed here below.

TABLE 1
The table includes information on T½ as measured in rats after intravenous administration of 15 nmol GH compound/rat as
described in Example 1.C. All compounds are based on GH (Q84C Y143C L101C) with the conjugate attached at L101C indicated by R1.

| GH | | |
|---|---|---|
| com- | | T½ |
| pound | Conjugate | (hours) |
| GH-A2 | 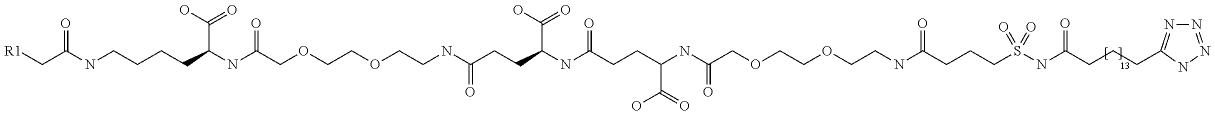 | 5.8 |
| GH-A3 | 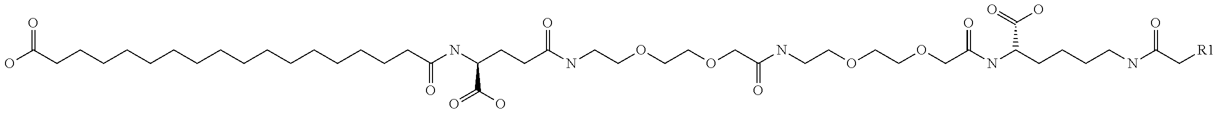 | 5.0 |
| GH-A4 | 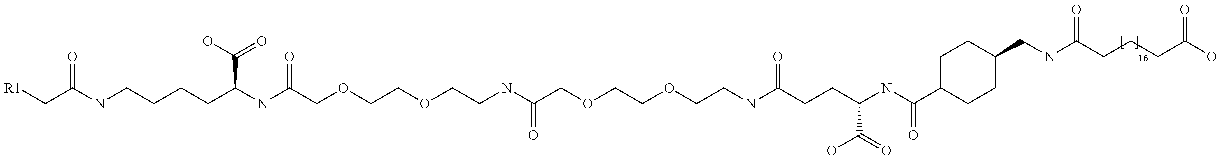 | 6.6 |
| GH-A5 | 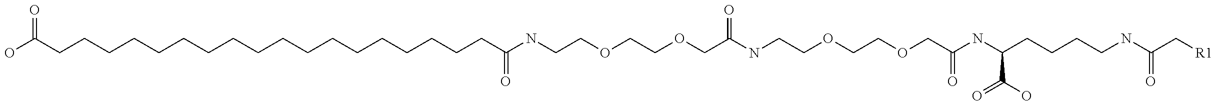 | 5.4 |
| GH-A6 | 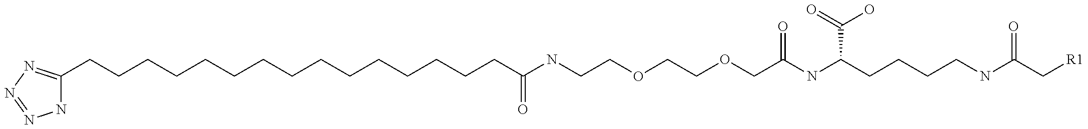 | 3.3 |
| GH-A7 | 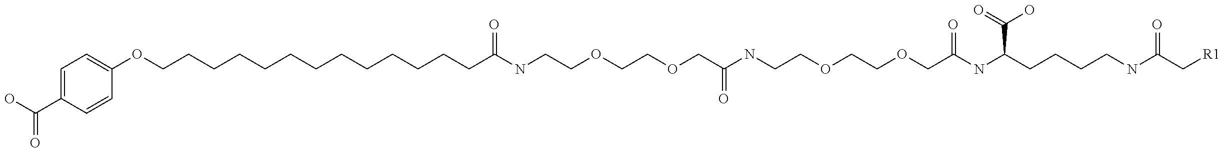 | 2.4 |
| GH-A8 | 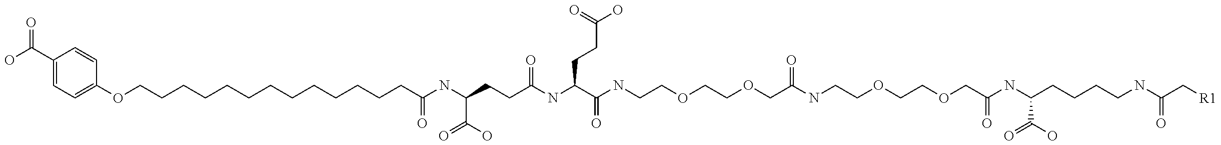 | 3.0 |
| GH-A9 | 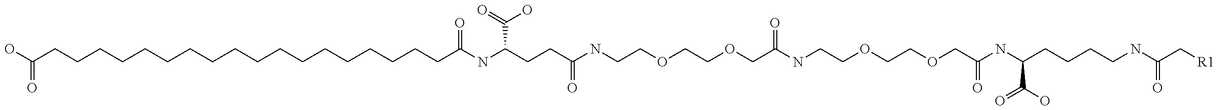 | 5.5 |

In one embodiment of the invention the growth hormone compound is dissolved and the pH of the resulting solution is adjusted to a value of the target pH value, which is 1 unit, alternatively 2 units and alternatively 2.5 pH units above or below the pl of the growth hormone compound, where after said resulting solution is freeze or spray dried. In one embodiment said pH adjustment is performed with a non-volatile acid or base.

The growth hormone compound may be present in an amount of a pharmaceutical composition according to the invention in up to about 20% such as up to about 10% by weight of the total pharmaceutical composition, or from about 0.1% such as from about 1%. In one embodiment of the invention, the growth hormone compound is present in an amount from about 0.1% to about 20%, in a further embodiment from about 0.1% to 15%, 0.1% to 10%, 1% to 8% or from about 1% to 5% by weight of the total composition. It is intended, however, that the choice of a particular level of growth hormone compound will be made in accordance with factors well-known in the pharmaceutical arts, including the solubility of the growth hormone compound in the solvent, e.g. an aqueous solution, polar organic solvent or optional hydrophilic component or surfactant used, or a mixture thereof, mode of administration and the size and condition of the patient.

Each unit dosage will suitably contain from 1 mg to 200 mg growth hormone compound, e.g. about 1 mg, 5 mg, 10 mg, 15 mg, 20 mg, 25 mg, 50 mg, 80 mg, 90 mg, 100 mg, 150 mg, 200 mg growth hormone compound, e.g. between 5 mg and 200 mg of growth hormone compound. In one embodiment of the invention each unit dosage contains between 10 mg and 200 mg of growth hormone compound. In a further embodiment a unit dosage form contains between 10 mg and 100 mg of growth hormone compound.

One embodiment of the invention, the unit dosage form contains between 20 mg and 80 mg of growth hormone compound. In yet a further embodiment of the invention, the unit dosage form contains between 30 mg and 60 mg of growth hormone compound.

In one embodiment of the invention, the unit dosage form contains between 30 mg and 50 mg of growth hormone compound. Such unit dosage forms are suitable for administration 1-5 times daily depending upon the particular purpose of therapy.

The production of polypeptides and peptides such as growth hormone is well known in the art. The growth hormone protein is usually produced by a method which comprises culturing a host cell containing a DNA sequence encoding the (poly)peptide and capable of expressing the (poly)peptide in a suitable nutrient medium under conditions permitting the expression of the peptide. For (poly)peptides comprising non-natural amino acid residues, the recombinant cell should be modified such that the non-natural amino acids are incorporated into the (poly)peptide, for instance by use of tRNA mutants.

### Formulation of Pharmaceutical Composition

Accordingly, one object of the invention is to provide a pharmaceutical formulation comprising a growth hormone compound in a therapeutically active amount. The concentration may vary from 0.25 mg/ml to 250 mg/ml in a solution or 2.5 mg/g to 250 mg/g in a solid dosage form.

Likewise the concentration of FA-aa may similarly vary from 0.25 mg/ml to 250 mg/ml in a solution or 2.5 mg/g to 250 mg/g in a solid dosage form.

In one embodiment the relative amount of the growth hormone compound and the FA-aa is from 10:1 to 1:10 (W:W), such as from 5:1 to 1:5 (W:W), 2:1 to 1:2 (W:W) or such as around 1:1 (W:W).

It is preferred that said formulation has a pH from 2.0 to 10.0. The formulation may further comprise a buffer system, a preservative, a tonicity agent, a chelating agent, a stabilizer, and/or a surfactant, as well as various combinations thereof. The use of preservatives, isotonic agents, chelating agents, stabilizers and surfactants in pharmaceutical compositions is well-known to the skilled person. Reference may be made to Remington: The Science and Practice of Pharmacy, 19th edition, 1995.

In one embodiment the pharmaceutical composition according to the present invention is a liquid.

In one embodiment, the pharmaceutical formulation is an aqueous formulation. Such a formulation is typically a solution or a suspension, but may also include colloids, dispersions, emulsions, and multi-phase materials. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment, the pharmaceutical formulation is a freeze-dried formulation, to which the physician or the patient adds solvents and/or diluents prior to use.

In another embodiment, the pharmaceutical formulation is a freeze-dried formulation which is filled into capsules.

In a further embodiment, the pharmaceutical formulation may be a semi-solid or solid formulation.

In one embodiment the oral pharmaceutical composition is a liquid. The liquid may non-the less be encapsulated or the like to enable that the oral pharmaceutical composition is swallowed before contact with the interior of the gastrointestinal system. This may be in the form of an enteric coating. As used herein the term “enteric coating” means a polymer coating that controls disintegration and release of the oral dosage form. The site of disintegration and release of the liquid dosage form may be designed depending on the pH of the targeted area, where absorption of the therapeutic protein is desired. Also include are thus acid resistant protective coatings and any other coating with enteric properties.

The term “enteric soft- or hard capsule technology” when used herein means soft- or hard capsule technology comprising at least one element with enteric properties, such as at least one layer of an enteric coating. The term “delayed release coatings” as used herein means a polymer coating which releases the API in a delayed manner after oral dosing. Delayed release can be achieved by pH dependent or pH independent polymer coatings.

Independent on formulation type a protease inhibitor may be included in the composition. Examples include SBTI, BBI and Chymostatin as examples of protease inhibitors, although alternative inhibitors may be desired for use as a pharmaceutical composition.

In one embodiment the FA-aa may be used in an aqueous liquid formulation, such liquid formulation comprising no organic solvents or below 10% of organic solvent. Such formulations are preferably buffered formulation, with a pH from around 4.0-10.0 such as from 5.0-9.0 such as from 5.5-8.5. The buffer may be selected from histidine, tris, HEPES, phosphate, glycine and carbonate buffers and mixtures thereof. The buffer may be selected from phosphate, glycine and carbonate buffers and mixtures thereof.

In further embodiments the formulation may be a phosphate buffered aqueous liquid with a pH around 6.0-8.0 or a glycine/Na-Bicarbonate buffered aqueous liquid with a pH around 8.0-8.5.

The concentration of the buffer may depend on the buffer used and may even vary for individual compounds to be formulated according to the invention. Phosphate buffers may be used at a concentration of 5-100 mM, such as 5-25 mM. Glycine may be used at a concentration of 5-50 mg/ml, preferably 20 mg/ml, whereas Na-Bicarbonate is used in concentration of 2.0-50 mg/ml, such as 2.0-5.0 mg/ml and preferably 2.4 mg/ml according to the present invention.

In one embodiment mannitol is included in the composition; D-mannitol may be used in concentration of 1-10 mg/ml, preferably around 2 mg/ml.

The oral pharmaceutical composition according to the invention may comprise further excipients. Reference may be made to Remington: The Science and Practice of Pharmacy, 19th edition, 1995.

In one embodiment the formulation may be non-aqueous, the term being used herein when referring to a composition to which no water is added during preparation of the pharmaceutical composition. It is known to the person skilled in the art that a composition which has been prepared without addition of water may take up small amounts of water from the surroundings during handling of the pharmaceutical composition such as e.g. a soft-capsule or a hard-capsule used to encapsulate the composition. Also, the growth hormone compound and/or one or more of the excipients in the pharmaceutical composition may have small amounts of water bound to it before preparing a pharmaceutical composition according to the invention. A non-aqueous pharmaceutical composition according to the invention may thus contain small amounts of water. In one embodiment a non-aqueous pharmaceutical composition according to the invention comprises less than 10% (w/w) water. In another embodiment, the composition according to the invention comprises less than 5% (w/w) water. In another embodiment, the composition according to the invention comprises less than 4% (w/w) water, in another embodiment less than 3% (w/w) water, in another embodiment less than 2% (w/w) water and in yet another embodiment less than 1% (w/w) water. In one embodiment the composition accord 0% (w/w) water.

In one embodiment the formulation may be “semi non-aqueous”, which mean that small amount of water is added during preparation of the pharmaceutical composition as for example a concentrated GH stock solution added to a non-aqueous formulation. In one embodiment a “semi non-aqueous” pharmaceutical composition according to the invention comprises less than 50% water. In another embodiment, the composition according to the invention comprises less than 40% (w/w) water. In another embodiment, the composition according to the invention comprises less than 30% (w/w) water. In another embodiment, the composition according to the invention comprises less than 20% (w/w) water. In another embodiment, the composition according to the invention comprises less than 15% (w/w) water. In another embodiment, the composition according to the invention comprises less than 10% (w/w) water.

The pharmaceutical composition may comprise a vegetable oil such as sesame oil.

The pharmaceutical composition may according to a further embodiment comprise a neutral oil such as Miglyol 810, Miglyol 829 or Miglyol 840.

The pharmaceutical composition according to any of the previous embodiments, wherein the composition further comprises a surfactant such as polysorbate 20, polysorbate 80, lauroglycol, capryol and/or labrasol.

In one embodiment the FA-aa may be used in an oil and surfactant based delivery system. In one embodiment the FA-aa may be used in a surfactant based formulation. The formulation may include polysorbate 20 (Tween 20), polysorbate 80 (Tween80), lauroglycol, capryol, labrasol and/or Span80 as surfactant.

In one embodiment the FA-aa may be used in a surfactant based delivery system. In one embodiment the FA-aa may be used in a liquid or semisolid liquid and surfactant based delivery system. In one embodiment the FA-aa may be used in a solid surfactant based delivery system. In one embodiment the FA-aa may be used in a solid oil and surfactant based delivery system.

In an embodiment the FA-aa may be used in a Self Emulsifying Drug Delivery Systems, also referred to as SEDDS, SMEDDS or SNEDDS composition.

In one embodiment the FA-aa may be used in a liquid, semisolid or solid surfactant based delivery system, such as SEDDS, SMEDDS or SNEDDS.

Liquid or semisolid SEDDS, SMEDDS or SNEDDS comprising FA-aa's according to the invention may be encapsulated with any available soft- or hard capsule technology to result in a solid oral pharmaceutical dosage form. Thus the term “solid” as used herein refers to liquid compositions encapsulated in a soft or hard capsule technology, but also to tablets and multi-particulates.

Liquid or semi-solid SEDDS, SMEDDS or SNEDDS comprising FA-aa's according to the invention may be encapsulated into porous microparticles. The porous microparticles may be filled into capsules or to be formulated into tablets. The capsules or tablets may be enteric coated for controlled delivery.

Liquid or semisolid SEDDS, SMEDDS or SNEDDS comprising FA-aa's according to the invention may be encapsulated with any available soft- or hard capsule technology to result in a solid oral pharmaceutical dosage form. In one embodiment of the invention the pharmaceutical composition is a SEDDS, SMEDDS or SNEDDS, comprising at least one growth hormone compound and at least one FA-aa. In further embodiment the composition may comprise propylene glycol and optionally additional components. In one embodiment the additional components may be at least one lipid and/or at least one surfactant.

In one embodiment the FA-aa may be used in a liquid or semisolid liquid and/or surfactant based delivery system, such as SEDDS, SMEDDS or SNEDDS.

In one embodiment the FA-aa may be used in a solid surfactant based delivery system, such as SEDDS, SMEDDS or SNEDDS.

In one embodiment pharmaceutical composition is a liquid or semisolid SEDDS, SMEDDS or SNEDDS comprising FA-aa's according to the invention and is encapsulated with any available soft- or hard capsule technology to result in a solid oral pharmaceutical dosage form.

In one embodiment a soft capsule technology used for encapsulating a composition according to the present invention is gelatine free. In one embodiment a gelatine free soft capsule technology as commercially known under the name Vegicaps® from Catalent® is used for encapsulation of the pharmaceutical composition according to the present invention.

In one embodiment a liquid or semisolid formulation according to the invention is encapsulated with any available soft- or hard capsule technology to result in a solid oral pharmaceutical dosage form further comprising enteric or delayed release coatings.

In one embodiment a liquid or semisolid formulation according to the invention is encapsulated with any available enteric soft- or hard capsule technology to result in a solid oral pharmaceutical dosage.

In one embodiment a liquid or semisolid SEDDS, SMEDDS or SNEDDS comprising FA-aa's according to the invention is encapsulated with any available soft- or hard capsule technology to result in a solid oral pharmaceutical dosage form further comprising an enteric or delayed release coatings. In one embodiment a liquid or semisolid SEDDS, SMEDDS or SNEDDS comprising FA-aa's according to the invention is encapsulated with any available enteric soft- or hard capsule technology to result in a solid oral pharmaceutical dosage.

In one embodiment a liquid or semisolid SEDDS, SMEDDS or SNEDDS comprising FA-aa's according to the invention is encapsulated with any available soft- or hard capsule technology to result in a solid oral pharmaceutical dosage form.

In one embodiment of the invention the pharmaceutical composition is a formulation, comprising at least one growth hormone protein or compound and at least one FA-aa and propylene glycol.

In one embodiment of the invention the pharmaceutical composition comprises of at least one growth hormone protein or compound and at least one FA-aa and propylene glycol.

In one embodiment of the invention the pharmaceutical composition is a SEDDS, SMEDDS or SNEDDS, comprising at least one peptide or protein and at least one FA-aa, propylene glycol.

In one embodiment the oral pharmaceutical composition comprises from 5 to 25% of propylene glycol.

In one embodiment, the oral pharmaceutical composition comprises at least one FA-aa, propylene glycol, and at least one non-ionic surfactant.

In one embodiment, the oral pharmaceutical composition comprises at least one FA-aa, propylene glycol, polysorbate 20 and a co-surfactant. Polysorbate 20 is a polysorbate surfactant whose stability and relative non-toxicity allows it to be used as a detergent and emulsifier in a number of domestic, scientific, and pharmacological applications. The number refers to the total number of oxyethylene —(CH₂CH₂O)— groups found in the molecule.

In one embodiment of the present invention, the oral pharmaceutical composition comprises at least one FA-aa, propylene glycol, polysorbate 20 and a polyglycerol fatty acid ester.

In one embodiment, the oral pharmaceutical composition comprises at least one FA-aa, propylene glycol, polysorbate 20 and a co-surfactant.

In one embodiment, the oral pharmaceutical composition comprises at least one FA-aa, propylene glycol, polysorbate 20 and a polyglycerol fatty acid ester such as diglycerol monocaprylate.

In certain embodiments of the present invention, the pharmaceutical composition may comprise additional excipients commonly found in pharmaceutical compositions, examples of such excipients include, but are not limited to, antioxidants, antimicrobial agents, enzyme inhibitors, stabilizers, preservatives, flavours, sweeteners and other components as described in Handbook of Pharmaceutical Excipients, Rowe et al., Eds., 4th Edition, Pharmaceutical Press (2003), which is hereby incorporated by reference

These additional excipients may be in an amount from about 0.05-5% by weight of the total pharmaceutical composition. Antioxidants, anti-microbial agents, enzyme inhibitors, stabilizers or preservatives typically provide up to about 0.05-2.5% by weight of the total pharmaceutical composition. Sweetening or flavouring agents typically provide up to about 2.5% or 5% by weight of the total pharmaceutical composition.

Oral pharmaceutical compositions according to this invention may be formulated as solid dosage forms.

Oral pharmaceutical compositions according to this invention may be formulated as solid dosage forms and may be selected from the group consisting of capsules, tablets, dragees, pills, lozenges, powders, extrudates or injection moulds and granules.

Oral pharmaceutical compositions according to this invention may be formulated as solid dosage forms and may be selected from the group consisting of capsules, tablets, dragees, pills, lozenges, powders and granules.

Oral pharmaceutical compositions according to this invention may be formulated as multi-particulate dosage forms.

Oral pharmaceutical compositions according to this invention may be formulated as multi-particulate dosage forms and may be selected from the group consisting of pellets, microparticles, nanoparticles, liquid or semisolid fill formulations in soft- or hard capsules, enteric coated soft or hard capsules.

In one embodiment the oral pharmaceutical compositions may be prepared with one or more coatings such as enteric coatings or be formulated as delayed release formulations according to methods well known in the art.

### Use of Pharmaceutical Composition

In one embodiment, the pharmaceutical composition according to the invention is used for the preparation of a medicament.

In one embodiment, the pharmaceutical composition according to the invention is used for the preparation of a medicament for the treatment or prevention of growth hormone deficiency in children and adults. Other diseases or disorders where an increased concentration of circulating growth hormone may be helpful may also be treated or prevented using the pharmaceutical composition of the invention. In one embodiment the pharmaceutical compositions of the invention is for treating diseases or states where a benefit from an increase in the amount of circulating growth hormone is observed. Such diseases or states include growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1st toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Chron's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; or short stature due to glucocorticoid treatment in children. Growth hormones have also been used for acceleration of the healing of muscle tissue, nervous tissue or wounds; the acceleration or improvement of blood flow to damaged tissue; or the decrease of infection rate in damaged tissue.

In one embodiment, the present invention relates to a method of treating diseases or states mentioned above, wherein the activity of the growth hormone compound is useful for treating said diseases or states. The administering of such compounds resulting in a therapeutic benefit associated with an increase in the amount of circulating growth hormone compound in the patient. In an embodiment said method comprises, administering to a patient an effective amount of a pharmaceutical composition of a growth hormone compound thereby ameliorating the symptoms of said patient.

In one embodiment, the present invention relates to a method comprising administration to a patient in need thereof an effective amount of a therapeutically effective amount of a pharmaceutical composition according to the invention comprising a growth hormone compound. The present invention thus provides a method for treating these diseases or states, the method comprising administering to a patient in need thereof a therapeutically effective amount of a growth hormone compound in a pharmaceutical composition according to the present invention.

A “therapeutically effective amount” of a compound of the invention as used herein means an amount sufficient to cure, alleviate or partially arrest the clinical manifestations of a given disease and its complications. An amount adequate to accomplish this is defined as “therapeutically effective amount”. Effective amounts for each purpose will depend on e.g. the severity of the disease or injury as well as the weight, sex, age and general state of the subject. It will be understood that determining an appropriate dosage may be achieved using routine experimentation, which is all within the ordinary skills of a trained physician or veterinary.

In one embodiment, the invention provides the use of a growth hormone compound or its conjugate in the manufacture of a medicament used in the treatment of the above mentioned diseases or states.

The present invention in further aspects relates to methods involving the pharmaceutical composition described herein above. The advantages of the pharmaceutical composition is to enable an increased up-take across the intestinal wall of a growth hormone compound compared to the up-take using a growth hormone composition not comprising any FA-aa's. The composition is considered to increase the permeability of the inner cell layer of the intestine.

A method for increasing the plasma concentration of a growth hormone compound comprising the step of exposing the gastrointestinal tract of an individual to an oral pharmaceutical composition as described herein above resulting in an increased plasma concentration of said growth hormone compound in said individual. As described above the oral pharmaceutical composition comprises in addition to the growth hormone compound at least one FA-aa.

A method for increasing the up-take of a growth hormone compound comprising the step of: exposing the gastrointestinal tract of an individual to a growth hormone compound and at least one FA-aa, whereby the plasma concentration of said growth hormone in said individual is increased compared to an exposure not including the at least one FA-aa.

As described herein exposure may be achieved by administering an oral pharmaceutical composition comprising in addition to the growth hormone compound at least one FA-aa.

A further aspect of the invention relates to a method for increasing the bioavailability of a growth hormone compound.

A further method according to the invention relates to a method for increasing uptake of a growth hormone compound across the epithelia cell layer of the gastro intestinal tract.

A further method according to the invention relates to a method for increasing up-take a growth hormone compound across the intestinal wall in an individual.

A further method is for increasing absorption of a growth hormone compound also involving the step as described above of administering a growth hormone compound and at least one FA-aa. In further embodiments the pharmaceutical compositions described herein may be used in any of the method described.

The invention is further illustrated, without being limited thereto, by the following embodiments.
1. A pharmaceutical composition comprising
   a. a growth hormone compound and
   b. at least one fatty acid amino acid (FA-aa) or a salt thereof.
2. The pharmaceutical composition according to embodiment 1, wherein the composition is an oral pharmaceutical composition.
3. The pharmaceutical composition according to embodiment 1 and 2, wherein the at least one fatty acid amino acid (FA-aa) is of the general formula:

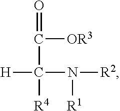

wherein
R1 is a fatty acid chain comprising 10 to 18 carbon atoms,
R2 is H (i.e. hydrogen), CH3 (i.e. methyl group) or a valence bond when R2 is covalently attached to R4, and
R3 is H or absent, and
R4 is an amino acid side chain, including —(CH₂)₃—, when covalently attached to R2.
   4. The pharmaceutical composition according to embodiment 3, wherein the amino acid R4 is selected from the groups of:
i. polar amino acid side chains,
ii. non-polar amino acid side chains
5. The pharmaceutical composition according to any of the previous embodiments, wherein the said FA-aa is in the form of a salt (R3 is absent).
6. The pharmaceutical composition according to any of the previous embodiments, wherein the said FA-aa is in the form of a salt with a monovalent cation, such as Na⁺ or K⁺.
7. The pharmaceutical composition according to any of the previous embodiments, wherein said FA-aa is in the form of its free acid (R3 is H).
8. The pharmaceutical composition according to any of the previous embodiments, wherein the fatty acid moiety of the FA-aa has at least 12 carbon atoms.
9. The pharmaceutical composition according to any of the previous embodiments, wherein the fatty acid moiety of the FA-aa has up to 18 carbon atoms.
10. The pharmaceutical composition to any of the previous embodiments, wherein the FA-aa comprises a fatty acid moiety of 12-18 carbon atoms.
11. The oral pharmaceutical composition according to any of the previous embodiments, wherein the, fatty acid moiety is palmitoyl derived from palmitic acid.
12. The pharmaceutical composition according to any of the previous embodiments, wherein the amino acid residue of said FA-aa is based on an amino acid selected from the group consisting of Aspartic acid (Asp), Glutamic acid (Glu), Sarcosine (Sarc), Glycine (Gly), Lysine (Lys), Arginine (Arg), Histidine (His), Glutamine (Gln), Asparagine (Asn), Serine (Ser), Threonine (Thr), Tyrosine (Tyr) and Cysteine (Cys).
13. The pharmaceutical composition according to any of the previous embodiments, wherein the amino acid residue of said FA-aa is based on an amino acid selected from the group consisting of Aspartic acid (Asp), Glutamic acid (Glu), Sarcosine (Sarc), Glycine (Gly), Glutamine (Gln), Asparagine (Asn), Serine (Ser) and Threonine (Thr).
14. The pharmaceutical composition according to any of the previous embodiments, wherein the amino acid residue of said FA-aa is based on an amino acid selected from the group consisting of Aspartic acid (Asp), Glutamic acid (Glu), Sarcosine (Sarc), Glycine (Gly), Glutamine (Gln) and Asparagine (Asn)
15. The pharmaceutical composition according to any of the previous embodiments, wherein the amino acid residue of said FA-aa is based on an amino acid selected from the group consisting of Aspartic acid (Asp), Glutamic acid (Glu, Glutamine (Gln), Asparagine (Asn) and Sarcosine (Sarc),
16. The pharmaceutical composition according to any of the previous embodiments, wherein the amino acid side chain R4 is —H (glycine).
17. The pharmaceutical composition according to any of the previous embodiments, wherein the amino acid residue is sarcosine (R4=—H (as glycine) and R1=—CH3).
18. The pharmaceutical composition according any of the previous embodiments, wherein the, fatty acid amino acid is N-palmitoyl-sarcosinate, sodium.
19. The pharmaceutical composition according any of the previous embodiments, wherein the fatty acid amino acid increases the permeability of HT29-MTX (E12) cells.
20. The pharmaceutical composition according any of the previous embodiments, wherein the growth hormone compound, wherein the protein sequence is at least 95% identical to human growth hormone (SEQ ID NO 1).
21. The pharmaceutical composition according any of the previous embodiments, wherein the growth hormone compound has a T ½ above 30 minutes.
22. The pharmaceutical composition according any of the previous embodiments, wherein the growth hormone compound comprises an additional di-sulfide bridge.
23. The pharmaceutical composition according any of the previous embodiments, wherein the growth hormone compound is a growth hormone derivative.
24. The pharmaceutical composition according any of the previous embodiments, wherein the growth hormone compound is a chemically modified growth hormone variant including an additional di-sulfide bridge.
25. The pharmaceutical composition according any of the previous embodiments, wherein the growth hormone compound is a chemically modified growth hormone, wherein the derivative is linked to an internal amino acid residue.
26. The pharmaceutical composition according any of the previous embodiments, wherein the growth hormone compound is chemically modified growth hormone comprising an acyl derivation.
27. The pharmaceutical composition according to any of the previous embodiments, wherein the composition is a liquid.
28. The pharmaceutical composition according to any of the previous embodiments, wherein the composition is an aqueous formulation.
29. The pharmaceutical composition according to any of the previous embodiments, wherein the composition is comprises less than 10% (w/w) water.
30. The pharmaceutical composition according to any of the previous embodiments, wherein the composition comprises further pharmaceutical excipients.
31. The pharmaceutical composition according to any of the previous embodiments, wherein the composition further comprises propylene glycol.
32. The pharmaceutical composition according to any of the previous embodiments, wherein the composition further comprises oil, such as a vegetable oil or neutral oil.
33. The pharmaceutical composition according to any of the previous embodiments, wherein the composition further comprises a mixture of oil and surfactant.
34. The pharmaceutical composition according to any of the previous embodiments, wherein the composition further comprises a surfactant such as polysorbate 20, polysorbate 80, lauroglycol, capryol or labrasol.
35. The pharmaceutical composition according to any of the previous embodiments, wherein the composition is a SEDDS, SMEDDS or SNEDDS formulation.
36. The pharmaceutical composition according to any of the previous embodiments, wherein the composition is a semi-solid composition
37. The pharmaceutical composition according to any of the previous embodiments, wherein the composition is a solid oral composition such as in the form of a tablet or a capsule.
38. The pharmaceutical composition according to any of the previous embodiments, further comprising a coating such as an enteric or delayed release coating.
39. The pharmaceutical composition according to any of the previous embodiments further comprising a protease inhibitor.
40. The pharmaceutical composition according to any of the previous embodiments further comprising a protease inhibitor.
41. The pharmaceutical composition according to any of the previous embodiments, for use as a medicament.
42. The pharmaceutical composition according to any of the previous embodiments, for use in treatment of a growth hormone related disease or disorder.
43. A method for increasing bioavailability of a growth hormone compound comprising a step of including a FA-aa in a pharmaceutical composition of a growth hormone compound administered to an individual.
44. A method for increasing the plasma concentration of a growth hormone compound comprising a step of exposing the gastrointestinal tract of an individual to a pharmaceutical composition comprising a growth hormone compound and a FA-aa resulting in an increased plasma concentration of said growth hormone compound in said individual.
45. The method of embodiment 44, wherein said exposure is achieved by oral administration of said pharmaceutical composition.
46. A method for increasing the up-take of a growth hormone compound comprising a step of exposing the gastrointestinal tract of an individual to a growth hormone compound and at least one FA-aa, whereby the plasma concentration of said growth hormone in said individual is increased compared to an exposure not including the at least one FA-aa.
47. A method for treatment of growth hormone relates diseases or disorders comprising administering a pharmaceutical composition comprising a growth hormone compound and at least one FA-aa.
48. A method for increasing uptake of a growth hormone compound across the intestinal wall comprising a step of, administering a pharmaceutical composition comprising a growth hormone compound and at least one FA-aa to an individual, whereby an increased uptake of said growth hormone compound is obtained compared to the uptake of said growth hormone compound obtained when said growth hormone composition does not including the at least one FA-aa.
49. The method of embodiment 44-48, where in the pharmaceutical composition is described by any one of embodiments 2-39.

### METHODS AND EXAMPLES

### Example 1

### A. General Method for Preparing a GH Compound

The gene coding for the growth hormone polypeptide is inserted recombinant into a plasmid vector. A suitable E. coli strain is subsequently transformed using the plasmid vector. hGH or GH variants may be expressed with an N-terminal methionine or as a MEAE fusion from which the MEAE sequence is subsequently cleaved off.

For the examples described here in a cell stock was prepared in 25% glycerol and stored at −80° C. Glycerol stock strain was inoculated into LB plates and subsequently incubated at 37° C. overnight. The content of each plate was washed with LB medium and diluted into 500 mL LB medium for expression. The cultures were incubated at 37° C. with shaking at 220 rpm until OD₆₀₀ 0.6 was reached. Succeeding induction was performed using 0.2 mM IPTG at 25° C. for 6 hours. Cells were finally harvested by centrifugation.

Cells were subsequently suspended in 10 mM Tris-HCl, pH=9.0 containing 0.05% Tween 20, 2.5 mM EDTA, 10 mM Cysteamine and 4M urea, and disrupted using a cell disrupter at 30 kPSI. The supernatant was collected by centrifugation and subsequently subjected to chromatographic purification.

The purification was performed using ion-exchange chromatography and hydrophobic interaction, followed by removal of the peptide tag using human dipeptidyl peptidase I (hDPPI) expressed from CHO cell. Final purification was achieved by isoprecipitation and ion-exchange chromatography.

The purification may also be achieved by using but not limited to ion-exchange chromatography, hydrophobic interaction chromatography, affinity chromatography, size exclusion chromatography and membrane based separation techniques known to a person skilled in the art.

### B. General Method for Testing Biological Activity of GH Compounds (BAF Assay)

The biological activity of GH compounds is measured in a cell based receptor potency proliferation assay, namely a BAF assay. The BAF-3 cells (a murine pro-B lymphoid cell line derived from the bone marrow) is IL-3 dependent for growth and survival. IL-3 activates JAK-2 and STAT which are the same mediators hGH is activating upon stimulation.

The BAF-3 cells were transfected with a plasmid containing the hGH receptor. Clones able to proliferate upon stimulation with hGH were turned into hGH-dependent cell lines hereafter referred to as BAF3-GHR. The cell lines respond to growth hormone with a dose-related growth pattern and can therefore be used to evaluate the effect of different GH compounds in a proliferation assay relative to hGH.

The BAF-3GHR cells are grown in starvation medium (culture medium without hGH) for 24 hours at 37° C., 5% CO2. The cells are centrifuged, the medium is removed and the cells are re-suspended in starvation medium to 2.22×105 cells/ml. Portions of 90 pl of the cell supernatant are seeded into microtiter plates (96 well NUNC-clone). Different concentrations of a given growth hormone compound are added to the cells, and the plates are incubated for 72 hours at 37° C., 5% CO2.

AlamarBlue™ (BioSource cat no Dal 1025) is a redox indicator, which is reduced by reactions innate to cellular metabolism and, therefore, provides an indirect measure of viable cell number. The AlamarBlue™ is diluted 6 times (5 pl AlamarBlue™+25 pl stavation medium) and 30 pl of the diluted AlamarBlue™ is added to each well. The cells are then incubated for another 4 hours. Finally the metabolic activity of the cells is measure in a fluorescence plate reader using an excitation filter of 544 nM and an emission filter of 590 nM. The result for a given compound is expressed as the ratio between EC50 of said compound and the EC50 of wt hGH run in parallel.

### C. General Method for Evaluating Pharmacokinetics Parameters of Growth Hormone Compounds

The pharmacokinetic of the compounds of the examples is investigated in male Sprague Dawley rats after intravenous (i.v.) and subcutaneous (s.c.) single dose administration.

Test compounds are diluted to a final concentration of 1 mg/mL in a dilution buffer consisting of: Glycine 20 mg/mL, mannitol 2 mg/mL, NaHCO₃ 2.5 mg/mL, pH adjusted to 8.2.

The test compounds are studied in male Sprague Dawley rats weighing 250 g. The test compounds are administered as a single injection either i.v. in the tail vein or s.c. in the neck with a 25 G needle at a predetermined dose such as of 15 nmol/rat in volume of 0.1 ml (concentration 150 nmol/ml) or 60 nmol/kg body weight.

For each test compound blood sampling may be conducted according to the schedule presented in table 2 below.

TABLE 2
Examplatory blood sampling schedule for investigating
pharmacokinetic of growth hormone compounds.

| Animal | | Sampling time (h) | | | | | | | | | | | | |
|---|---|---|---|---|---|---|---|---|---|---|---|---|---|---|
| no. | RoA | Predose | 0.08 | 0.25 | 0.5 | 1 | 2 | 4 | 6 | 8 | 18 | 24 | 48 | 72 |
| 1 | s.c. | | | | | | | X | X | X | | X | X | X |
| 2 | | | | | | | | X | X | X | | X | X | X |
| 3 | | | | X | X | X | X | | | | | | | |
| 4 | | | | X | X | X | X | | | | | | | |
| 5 | | | X | | | | | | | | X | | | |
| 6 | | | X | | | | | | | | X | | | |
| 7 | i.v. | | | | X | X | | | X | X | | X | X | X |
| 8 | | | | | X | X | | | X | X | | X | X | X |
| 9 | | X | X | | | | | | | | X | | | |
| 10 | | X | X | | | | | | | | X | | | |

Values in table 1 was obtained with an alternative blood sampling schedule including sampling at pre dose, 0.08 h, 0.5 h 1 h 2 h 4 h 7 h 18 h 24 h 48 h 72 h and 96 h.

At each sampling time 0.25 ml blood is drawn from the tail vein using a 25 G needle. The blood is sampled into a EDTA coated test tube and stored on ice until centrifugation at 1200×G for 10 min at 4° C. Plasma is transferred to a Micronic tube and stored at −20° C. until analysis.

Test compound concentrations are determined by a sandwich ELISA using a guinea pig anti-hGH polyclonal antibody as catcher, and biotinylated hGH binding-protein (soluble part of human GH receptor) as detector. The limit of detection of the assay was 0.2 nM.

A non-compartmental pharmacokinetic analysis is performed on mean concentration-time profiles of each test compound using WinNonlin Professional (Pharsight Inc., Mountain View, Calif., USA). The pharmacokinetic parameter estimates of terminal half-life (t_{1/2}) and mean residence time (MRT) are calculated.

### D. Method for Measuring Transepithelial Transport of Growth Hormone Compounds In Vitro, Using Monolayers of E12 Cells

### Cell Culturing

HT29-MTX (E12) cells (Pharm Res. 2001; 18(8):1138-45 and Pharm Res. 2007; 24(7):1346-56) were grown in Dulbecco's Modified Eagle Medium supplemented with 10% foetal bovine serum (FBS), 1% penicillin/streptomycin, 1% L-glutamine and 1% non-essential amino acids. For the transport assay, E12 cells were seeded onto tissue culture treated polycarbonate filters in 12-well Transwell® plates (1.13 cm2, 0.4 μm pore size) at a density of 10⁵ cells per well. Cells were cultured at 37° C. in an atmosphere of 5% CO₂ and the culture media was exchange every second day. Transport experiments were performed after 14-18 days in culture.

### Trans-Epithellal Transport

The amount of GH compound transported from the donor chamber (apical side) to the receiver chamber (basolateral side) was measured.

Before the experiment, the E12 cells are equilibrated for 60 min with transport buffer on both sides of the epithelium. Buffer is then removed and the experiment initiated by adding a test solution to the donor chamber and a corresponding transport buffer to the receiver chamber. Donor samples (20 pl) are taken at 0 min and at the end of the experiment. Receiver samples (200 pl) are taken at regular intervals such as every 15 min. The study is usually performed in an atmosphere of 5% CO2-95% O₂ at 37° C. on a shaking plate (30 rpm).

The apparent permeability (Papp) is measured as the amount of GH transferred across the membrane in a certain period relative to the initial concentration and the area of the membrane (Flux/(area*initial conc.). The concentration of a GH compound may be determined by a sandwich ELISA using a guinea pig anti-hGH polyclonal antibody as catcher, and biotinylated hGH binding-protein (soluble part of human GH receptor) as detector. The limit of detection of the assay was 0.2 nM.assay.

Transport of [3H]mannitol, a marker for paracellular transport, may be measured, to verify the integrity of the epithelium, using a scintillation counter.

Before and during the experiment the transepithelial electrical resistance (TEER) of the cell monolayers is monitored. The TEER is measured with EVOM™ Epithelial Voltohmmeter connected to Chopsticks.

### Example 2

### Preparation of Albumin binder 4-(1H-Tetrazol-16-yl-hexadecanoylsulfamoyl)butanoyl-OEG-gammaGlu-gammalGlu-OEG-N^{ε}(C(O)CH₂Br)Lys-OH

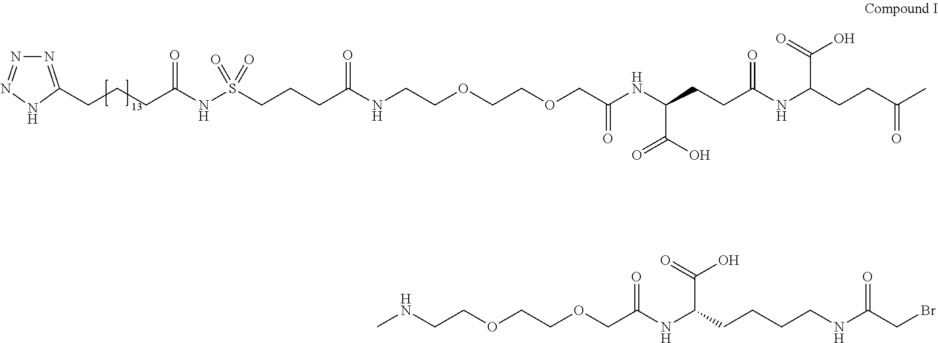

The compound I was synthesised on solid support in 1 mM scale using standard Fmoc-peptide chemistry on ABI433 synthesizer. Peptide was assembled on a Fmoc-Lys(MTT)-Wang resin using Fmoc-OEG-OH and Fmoc-Glu-OtBu protected amino acids. 4-(16-1H-Tetrazol-5-yl-hexadecanoylsulfamoyl)butyric acid was manual coupled with DIC/NHS in DCM/NMP, 2 eq. overnight, TNBS test showed the reaction to be complete. The resin was then treated with 50 mL DCMITFA/TIS/water (94:2:2:2) in a flow through arrangement until the yellow colour disappeared, ˜20 min. followed by washing and neutralizing with DIPEA/DMF. Bromo acetic acid (4 mM) in DCM/NMP (1:1) was activated with a 1 mM mixture of HONSu and DIC. Filtered and added to the resin with addition of further 1 mM of DIPEA. After 1 hr the reaction was completed. The resin was treated with 80 mL TFA/TIS/water (95:2.5:2.5) for 1 hr. Evaporated with a stream of N₂, precipitated by addition of Et₂O and washed with Et₂O and dried. Crude product was purified on preparative HPLC (2 runs), with a gradient from 30-80% 0.1 TFA/MeCN against 0.1% TFA in water. Fractions were collected and lyophilized with ˜50% MeCN affording compound I.

TOF-MS: mass 1272.52 (M+1)

### Example 3

### Preparation of Albumin Binder

In a similar way as described in Example 2 above the following compound was prepared using Fmoc-Lys(Mtt)-OH and Wang Resin.

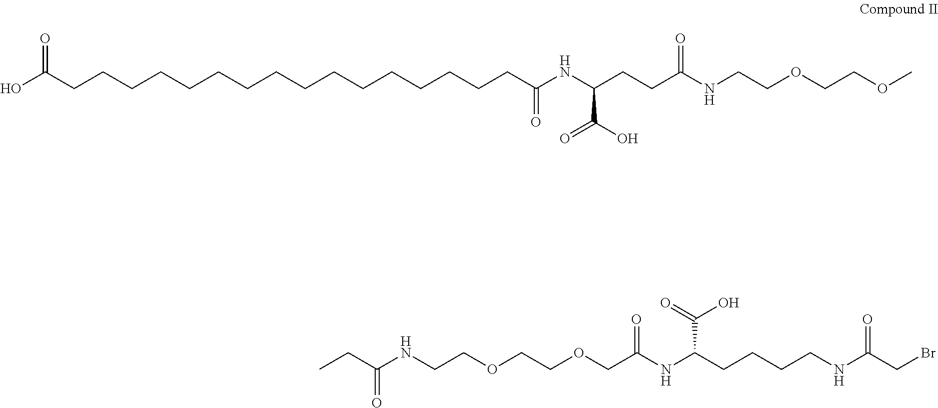

TOF-MS: mass 983.01 (M+1)

### Example 4

### Conjugation of Albumin Binder (Compound II) to hGH(Q84C Y143C L101C)

To a 310 ml solution of 4.9 mg/ml hGH(Q84C Y143C L101C)-cysteamine in 20 mM Tri-ethanol amine, 0.1 M NaCl, pH 8.5 was added 387 mg TSPP (Tris(3-sulfonatephenyl)phosphine hydrate sodium salt) by mixing at ambient temperature. The reaction was allowed to run for two hours.

The albumin binder compound II was dissolved in 20 mM Tri-ethanol amine, 2 mM EDTA pH 8.5 to 10 mg/ml. The dissolved albumin binder was added to the deprotected hGH[Q84C Y143C L101C] in a ratio of 3:1. NaCl was added to the solution to obtain a final concentration of NaCl of 0.4 M. The reaction was run at ambient temperature for 2.5 hours and subsequently placed at 4 degrees Celsius over night until final purification was performed, affording compound GH-A3.

### Example 5

Conjugation of albumin binder (compound I) to hGH(Q84C Y143C L101C) was done in a similar way as described in example 4, affording compound GH-A2.

### Example 6

Conjugated compounds GH-A2 and GH-A3 were subsequently purified using ion-exchange chromatography followed by buffer exchange performed as diafiltration. The purification could also be achieved by using but not limited to ion-exchange chromatography, hydrophobic interaction chromatography, affinity chromatography, size exclusion chromatography and membrane based separation techniques known to a person skilled in the art.

Growth hormone compound 1 (GH-A1): GH(Q84C Y143C)

Growth hormone compound 2 (GH-A2): GH(Q84C Y143C L101C) with albumin binder

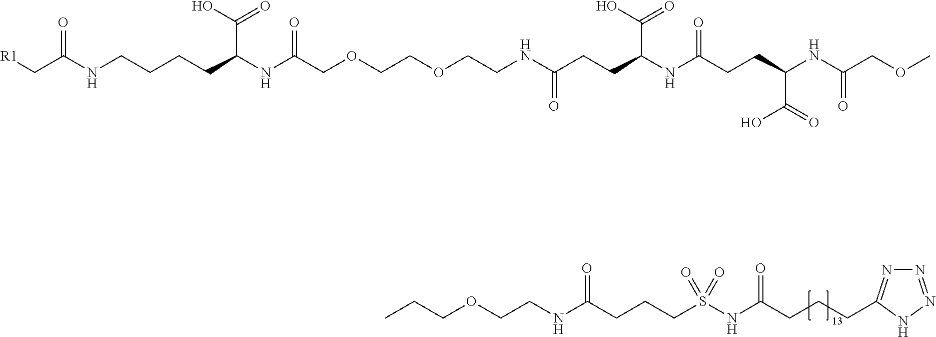

Albumin binder of GH-A2

R1=S of L101C in hGH(Q84C Y143C L101C)

Growth hormone compound 3 (GH-A3): GH(Q84C Y143C L101C) with albumin binder

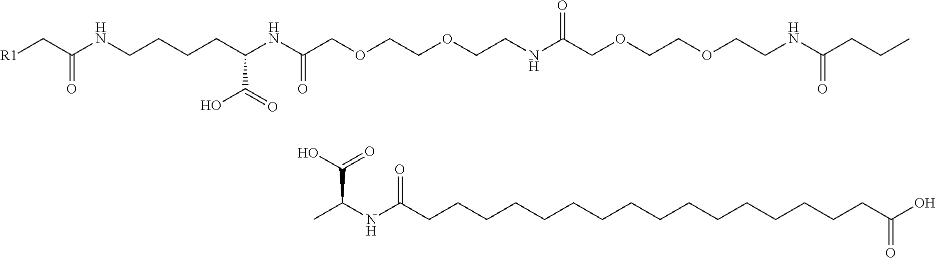

Albumin binder of GH-A3

R1=S of L101C in hGH[Q84C Y143C L101C)

### Example 7

### Protein Chemical Characterization of Purified Growth Hormone Compounds

The intact purified protein was analysed using MALDI-MS. The observed mass corresponded to the theoretical mass deduced from the amino acid sequence. Linking disulfide bonds may be demonstrated by peptide mapping using trypsin and AspN digestion followed by MALDI-MS analysis of the digest before and after reduction of the disulfide bonds with DTT.

### Concentration Determination of the Purified Growth Hormone Compounds

RP-HPLC analysis was performed on a Agilent 1100 system using a Vydac 218TP54 4.6 mm×250 mm 5 μm C-18 silica column (The Separations Group, Hesperia). Detection was by UV at 214 nm. The column was equilibrated with 0.1% trifluoracetic acid/H₂O and the sample was eluted by a suitable gradient of 0 to 90% acetonitrile against 0.1% trifluoracetic acid/H₂O. A calibration curve using a hGH standard of known concentration was used to calculate the concentration of the hGH compound sample.

Protein concentrations were estimated by measuring absorbance at 280 nm using a NanoDrop ND-1000 UV-spectrophotometer.

Size exclusion chromatography was performed on a Agilent 1100 system using a TSK2000 SWXL 7.5×300 mm column (Tosoh). Detection was by UV at 215 nm. The column was equilibrated with 0.063 M Phosphate Buffer (7.77 g Na2HPO4 2H2O and 5.28 g NaH2PO4H2O to 1 l mobile phase) containing 3% isopropanol (30 ml to 11 mobile phase) pH 7.0 (adjusted with phosphoric acid) which was also used as elution buffer. For each sample 20 ul was injected on the column. A calibration curve using a hGH standard of known concentration was used to calculate the concentration of the hGH compound sample.

Amount of monomeric, dimeric and multimeric protein was determined as percentage of the total area under the curve.

A list of short names, long names and IUPAC names of compound used is provided below.

| Short name | Long Name | IUPAC Name |
|---|---|---|
| C8-His | N-octanoate-L- | sodium (2S)-3-(1H-imidazol-4-yl)-2- |
| | histidine | (octanoylamino)propanoate |
| C8-Sarc | N-octanoyl- | sodium (N-methyl-octanoyl-amino)acetate |
| | sarcosine | |
| C8-Leu | N-octanoyl-L- | sodium (2S)-2-(octanoylamino)-4- |
| | leucine | methylpentanoate |
| C10-Asp | N-decanoyl-L- | disodium (2S)-2-(decanoylamino)butanedioate |
| | aspartic acid | |
| C10-Gln | N-decanoyl-L- | sodium (2S)-5-amino-2-(decanoylamino)-5- |
| | glutamine | oxopentanoate |
| C10-Gly | N-decanoyl- | sodium 2-(decanoylamino)acetate |
| | glycine | |
| C10-Leu | N-decanoyl-L- | sodium (2S)-2-(decanoylamino)-4- |
| | leucine | methylpentanoate |
| C12-Glu | N-lauroyl-L- | disodium (2S)-2-(dodecanoylamino)pentanedioate |
| | glutamic acid | |
| C12-His | N-lauroyl-L- | sodium (2S)-2-(dodecanoylamino)-3-(1H-imidazol- |
| | histidine | 4-yl)propanoate |
| C12-Leu | N-dodecanoyl-L- | sodium (2S)-2-(dodecanoylamino)-4- |
| | leucine | methylpentanoate |
| C12-Pro | N-lauroyl-L-proline | sodium (2S)-1-dodecanoylpyrrolidine-2- |
| | | carboxylate |
| C12-Sarc | N-lauroyl-L- | sodium (N-methyl-dodecanoyl-amino)acetate |
| | sarcosine | |
| C~12-Sarc | N-cocoyl- | sodium (N-methyl-dodecanoyl-amino)acetate |
| | sarcosine | (main component) |
| C12-Trp | N-lauroyl-L- | sodium (2S)-2-(dodecanoylamino)-3-(1H-indol-3- |
| | tryptophan | yl)propanoate |
| C14-Gln | N-tetradecanoyl-L- | sodium (2S)-5-amino-5-oxo-2- |
| | glutamine | (tetradecanoylamino)pentanoate |
| C14-Glu | N-tetradecanoyl-L- | disodium (2S)-2- |
| | glutamine | (tetradecanoylamino)pentanedioate |
| C14-DGlu | N-myristoyl-D- | disodium (2R)-2- |
| | glutamic acid | (tetradecanoylamino)pentanedioate |
| C14-Sarc | N-myristoyl- | sodium (N-methyl-tetradecanoyl-amino)acetate |
| | sarcosine | |
| C16-Gln | N-palmitoyl-L- | sodium (2S)-5-amino-5-oxo-2- |
| | glutamine | (hexadecanoylamino)pentanoate |
| C16-Glu | N-palmitoyl-L- | disodium (2S)-2- |
| | glutamic acid | (hexadecanoylamino)pentanedioate |
| C16-Sarc | N-palmitoyl- | sodium 2-[hexadecanoyl(methyl)amino]acetate |
| | sarcosine | sodium (N-methyl-hexadecanoyl-amino)acetate |
| C=18-Sarc | N-oleoyl sarcosine | sodium 2-[methyl-[(E)-octadec-9- |
| | | enoyl]amino]acetate |
| | | sodium [N-methyl-((E)-octadec-9-enoyl)- |
| | | amino]acetate |

### Example 8

### Intra Intestinal Administration of GH Compounds to Rats and Analysis of Blood Samples

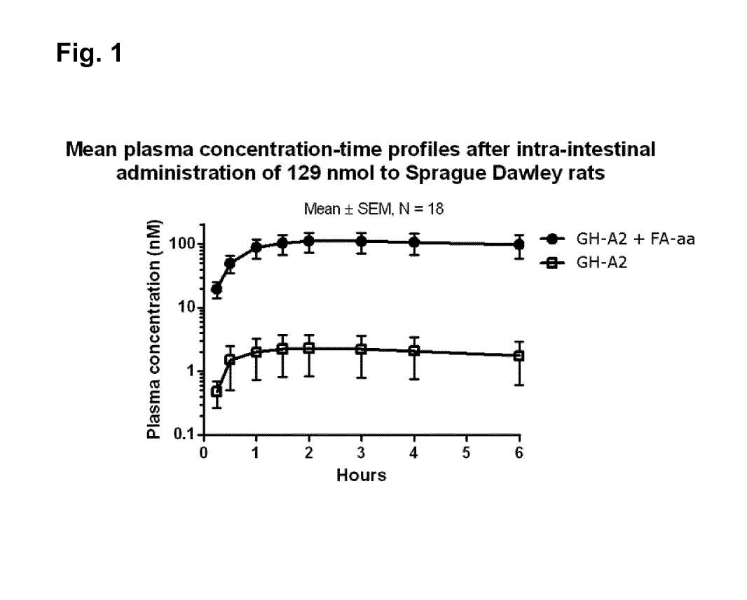

Growth hormone compounds (129/130, 150 or 300 nmol/rat) were formulated according to examples 9-19 and results presented in tables A-K and FIGS. 1-7. Each composition is prepared as a 100 pl dosage injected into mid-jejenum of fully anaesthetized overnight fasted Sprague-Dawley rats (n=6, 9 or 18). The concentration of GH to FA-aa adjusted around 1:1 (W:W) but variations from 6:1 to 1:2 are included.

At each sampling time 0.25 ml blood is drawn from the tail vein using a 25 G needle. Sampling times are: pre-dose and 0.25, 0.5, 1, 2, 4 and 6 hours after dosing. The blood is sampled into a EDTA coated test tube and stored on ice until centrifugation at 1200×G for 10 min at 4° C. Plasma is transferred to a Micronic tube and stored at −20° C. until analysis.

Test compound concentrations are determined by a sandwich ELISA using a guinea pig anti-hGH polyclonal antibody as catcher, and biotinylated hGH binding-protein (soluble part of human GH receptor) as detector. The limit of detection of the assay was 0.2 nM.

A non-compartmental pharmacokinetic analysis is performed on mean concentration-time profiles of each test compound using WinNonlin Professional (Pharsight Inc., Mountain View, Calif., USA). The pharmacokinetic parameter estimates of terminal half-life (t_{1/2}) and mean residence time (MRT) are calculated.

The samples were analysed using a Luminescence Oxygen Channeling Immunoassay (LOCI), a homogenous bead based assay. LOCI reagents include two latex bead reagents and biotinylated GH binding protein, which is one part of the sandwich. One of the bead reagents is a generic reagent (donor beads) and is coated with streptavidin and contains a photosensitive dye. The second bead reagent (acceptor beads) is coated with an antibody making up the sandwich. During the assay the three reactants combine with analyt to form a bead-aggregate-immune complex. Illumination of the complex releases singlet oxygen from the donor beads which channels into the acceptor beads and triggers chemiluminescence which is measured in the EnVision plate reader. The amount of light generated is proportional to the concentration of hGH derivative. The pharmacokinetic profile was retrieved from the resulting records.

An example of such a pharmacokinetic profile is shown in FIG. 1. The enhancer tested in the shown example is C16-Glu and the plasma concentration of growth hormone compound 2 (GH-A2) was measured after administration with or without the enhancer as described above.

The degree of GH absorption is quantified as AUC (area under the curve) based on the pharmacokinetic profile. In the following examples the degree of enhancement has been calculated as AUC in presence of enhancing formulation divided by AUC in absence of enhancing formulation. The effect of C16-Glu as shown in FIG. 1 is also covered by Example 11.

### Example 9

An aqueous formulation of growth hormone containing fatty acid acylated amino acids was tested. Growth hormone compound 1 (GH-A1) (300 nmol/rat) dissolved in 10 mM sodium phosphate buffer, pH 6.5 in presence of fatty acid acylated amino acids. The composition was injected into mid-jejunum of anaesthetized overnight fasted Sprague-Dawley rats (n=6) and the pharmacokinetic profile was retrieved as described in example 8 and the degree of enhancement calculated. The results are shown in Table A.

TABLE A

| Fatty acid amino acid | Concentration of FA-aa | |
|---|---|---|
| (FA-aa) | (mg/ml) | AUCₑₙₕ/AUC_{no enh} |
| C16-Sarc | 30 | 6 |

### Example 10

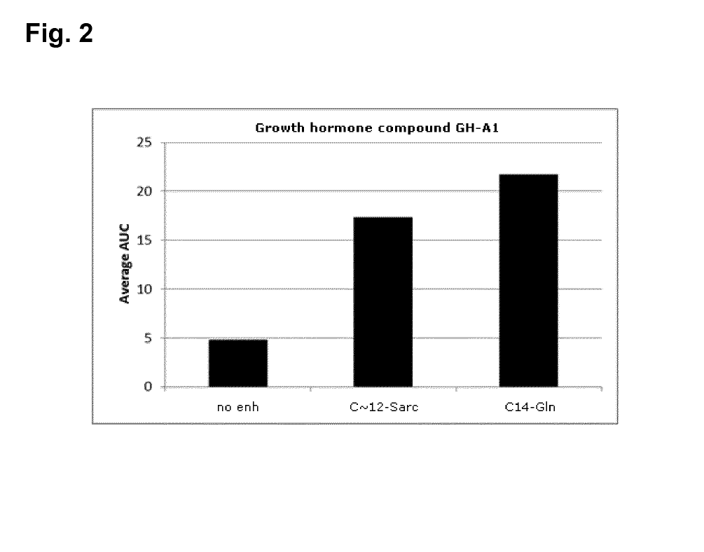

Aqueous formulations of growth hormone containing fatty acid acylated amino acids were tested. Growth hormone compound GH-A1 (300 nmol/rat) dissolved in buffer: 20 mg/ml Glycine, 2 mg/ml D-Mannitol, 2.4 mg/ml Na-Bicarbonate, pH=8.2, in presence of fatty acid acylated amino acids. The composition was injected into mid-jejunum of anaesthetized overnight fasted Sprague-Dawley rats (n=6) and the pharmacokinetic profile was retrieved and the degree of enhancement calculated as described in example 8. The results are shown in Table B. Additional studies were performed using the same FA-aa and GH compound using alternative buffers including (e.g. 20 mg/ml Glycine, 2 mg/ml D-Mannitol, 2.4 mg/ml Na-Bicarbonate, pH=8.2; 50 mM sodium phosphate buffer pH 7.5; 10 mM sodium phosphate buffer pH 6.5 and 50 mM sodium phosphate buffer pH 6). Averages of the obtained results are presented in FIG. 2.

TABLE B

| Fatty acid amino acid | Concentration of FA-aa | |
|---|---|---|
| (FA-aa) | (mg/ml) | AUCₑₙₕ/AUC_{no enh} |
| C14-Gln | 10 | 5 |
| C~12-Sarc | 30 | 4 |

### Example 11

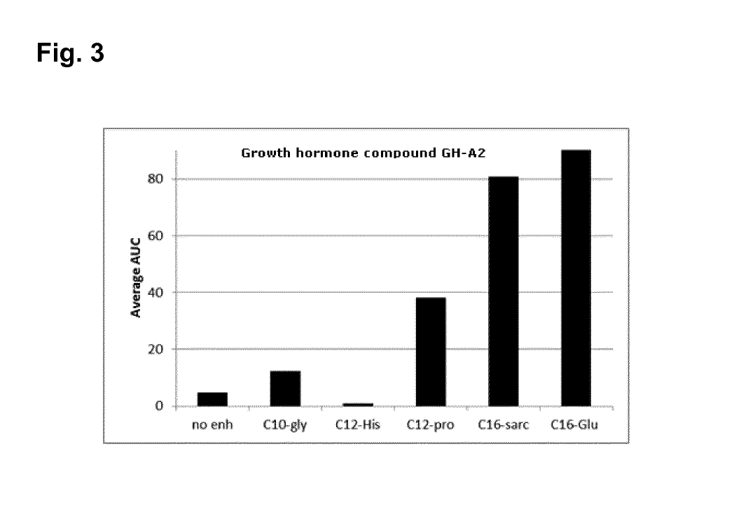

Aqueous formulations of growth hormone containing fatty acid acylated amino acids were tested. Growth hormone compound GH-A2 (150 nmol/rat) dissolved in buffer: 20 mg/ml Glycine, 2 mg/ml D-Mannitol, 2.4 mg/ml Na-Bicarbonate, pH=8.2, in presence of fatty acid acylated amino acids. The composition was injected into mid-jejunum of anaesthetized overnight fasted Sprague-Dawley rats (n=6) and the pharmacokinetic profile was retrieved and the degree of enhancement calculated as described in example 8. The results are shown in Table C. Additional studies were performed using the same FA-aa and GH compound using alternative buffers including (e.g. 20 mg/ml Glycine, 2 mg/ml D-Mannitol, 2.4 mg/ml Na-Bicarbonate, pH=8.2 and 10 mM sodium phosphate buffer pH 6.5). Averages of the obtained results are presented in FIG. 3, while FIG. 1 includes data from a repetition of the experiment with n=18.

TABLE C

| Fatty acid amino acid | Concentration of FA-aa | |
|---|---|---|
| (FA-aa) | mg/ml | AUCₑₙₕ/AUC_{no enh} |
| C16-Glu | 30 | ~200 |
| C16-Sarc | 30 | 18 |
| C12-Pro | 30 | 8 |
| C10-Gly | 30 | 3 |
| C12-His | 30 | 0.4 |

### Example 12

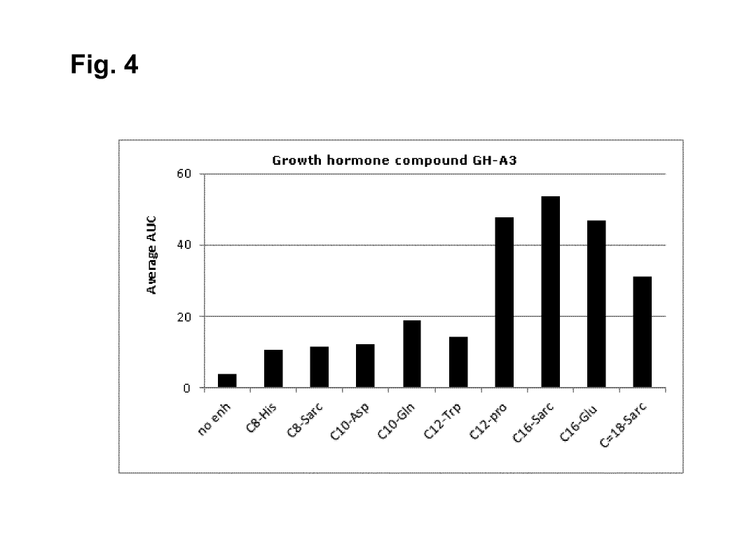

Aqueous formulations of growth hormone containing fatty acid acylated amino acids were tested. Growth hormone compound GH-A3 (300 nmol/rat) dissolved in buffer: 20 mg/ml Glycine, 2 mg/ml D-Mannitol, 2.4 mg/ml Na-Bicarbonate, pH=8.2, in presence of fatty acid acylated amino acids. The composition was injected into mid-jejunum of anaesthetized overnight fasted Sprague-Dawley rats (n=6) and the pharmacokinetic profile was retrieved and the degree of enhancement calculated as described in example 8. The results are shown in Table D. Additional studies were performed using the same FA-aa and GH compound using alternative buffers. Averages of the obtained results are presented in. Additional studies were performed using the same FA-aa and GH compound using alternative buffers including 20 mg/ml Glycine, 2 mg/ml D-Mannitol, 2.4 mg/ml Na-Bicarbonate, pH=8.2; 50 mM sodium phosphate, 50 mM NaCl, pH 8; and 10 mM sodium phosphate buffer pH 6.5. Averages of the obtained results are presented in FIG. 4.

TABLE D

| Fatty acid amino acid | Concentration of FA-aa | |
|---|---|---|
| (FA-aa) | (mg/ml) | AUCₑₙₕ/AUC_{no enh} |
| C=18-Sarc | 30 | 8 |
| C16-Sarc | 30 | 29 |
| C16-Glu | 30 | 12 |
| C12-Pro | 30 | 12 |
| C12-Trp | 30 | 4 |
| C10-Asp | 30 | 3 |
| C10-Gln | 30 | 4 |
| C8-His | 30 | 3 |
| C8-Sarc | 30 | 3 |

The in vivo data from examples 9-12 demonstrates that all enhancers increase the AUC of the GH compound compared to the AUC of the GH compound measured in the absence of enhancer. The data further suggests that degree of GH compound adsorption is influenced by the identity of the fatty acid acylated amino acids. The strongest effect is observed for FA-aa's with longer fatty acid chains and the effects further, to some degree, depend on the specific amino acid.

### Example 13

An aqueous formulation of growth hormone containing a fatty acid acylated amino acid and a protease inhibitor was tested. Growth hormone compound GH-A2 (150 nmol/rat) was dissolved in 10 mM sodium phosphate buffer pH 8.2 in presence of fatty acid acylated amino acids and Soy Bean Trypsin Inhibitor (SBTI) 2%. The composition was injected into mid-jejunum of anaesthetized overnight fasted Sprague-Dawley rats (n=12) and the pharmacokinetic profile was retrieved and the degree of enhancement calculated as described in example 8. The results are shown in Table E.

TABLE E

| Fatty acid amino | Concentration | Concentration of | AUCₑₙₕ/ |
|---|---|---|---|
| acid (FA-aa) | of FA-aa mg/ml | inhibitor | AUC_{no enh} |
| C16-Sarc | 30 | 2% | 36 |

### Example 14

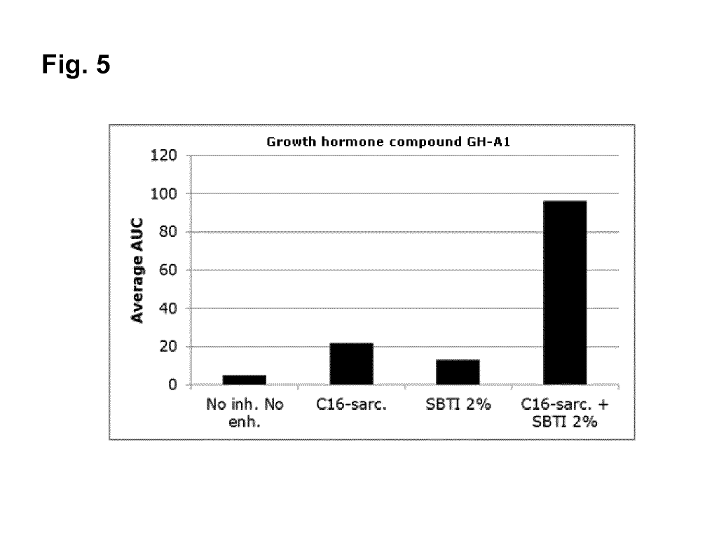

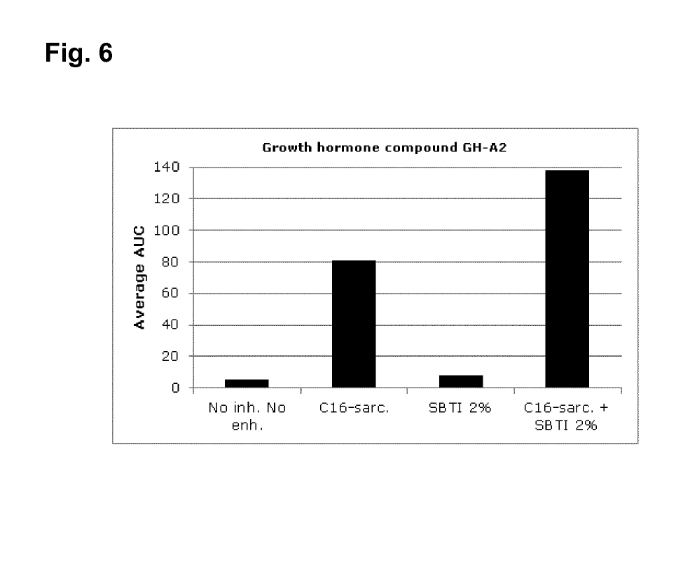

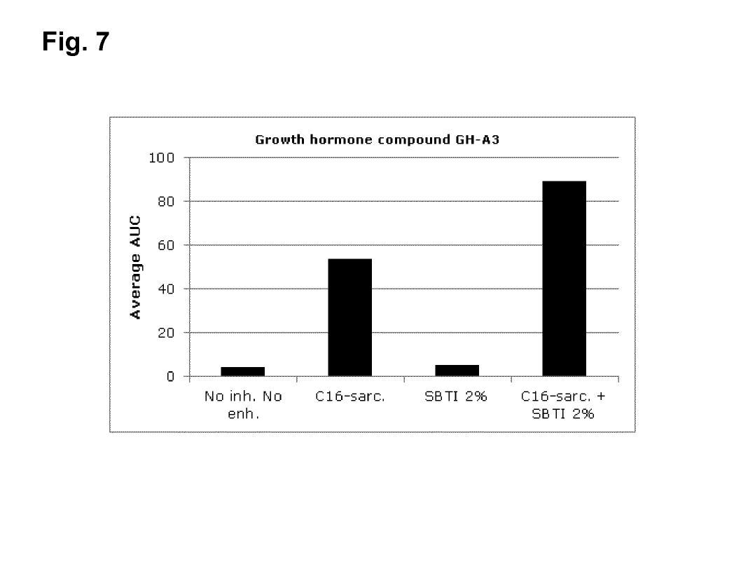

Aqueous formulations of growth hormones containing fatty acid acylated amino acid and protease inhibitor were tested. Growth hormone compound GH-A1, GH-A2 and GH-A3 (150 nmol/rat) dissolved in 10 mM sodium phosphate buffer pH 6.5 in presence of fatty acid acylated amino acids and Soy Bean Trypsin Inhibitor (SBTI) 2%. The composition was injected into mid-jejunum of anaesthetized overnight fasted Sprague-Dawley rats (n=6-12) and the pharmacokinetic profile was retrieved and the degree of enhancement calculated as described in example 8. The results are shown in Table F. Additional studies were performed using the same FA-aa and GH compounds using alternative buffers. The aqueous buffers tested includes 20 mg/ml Glycine, 2 mg/ml D-Mannitol, 2.4 mg/ml Na-Bicarbonate, pH=8.2; 10 mM sodium phosphate, pH 8.2 and 10 mM sodium phosphate, pH 6.5. Averages of obtained results using GH-A1, GH-A2 and GH-A3, FA-aa's and inhibitors are presented in FIG. 5, FIG. 6 and FIG. 7, respectively.

TABLE F

| | Fatty | Concentration | Amount of | |
|---|---|---|---|---|
| GH | acid amino | of FA-aa | protease | AUCₑₙₕ/ |
| compound | acid (FA-aa) | (mg/ml) | inhibitor | AUC_{no enh} |
| GH-A1 | C16-Sarc | 30 | 2% | 20 |
| GH-A2 | C16-Sarc | 30 | 2% | 26 |
| GH-A3 | C16-Sarc | 30 | 2% | 23 |

The in vivo data suggests that the GH adsorption enhancing effect of the fatty acid acylated amino acid (here C16-sarcosinate) is significantly improved by the presence of an enzyme inhibitor. As seen in FIGS. 5, 6 and 7 the effect of adding the proteinase inhibitor (SBTI) in combination with a FA-aa enhancer is more than additive for all three Growth hormone compounds.

### Example 15

Non-aqueous formulations of growth hormone containing fatty acid acylated amino acids were tested. A mixture of a vegetable oil and a surfactant in weight ratios 30-50 wt % oil and 50-70 wt % surfactant is used as solvent for growth hormone compound 2 in presence of fatty acid acylated amino acids. The composition (150 nmol/rat) was injected into mid-jejunum of anaesthetized overnight fasted Sprague-Dawley rats (n=8) and the pharmacokinetic profile was retrieved and the degree of enhancement calculated as described in example 8. The results are shown in Table G.

TABLE G

| | Fatty acid | | |
|---|---|---|---|
| Non-aq Formulation | amino acid | Concentration of | AUCₑₙₕ/ |
| (weight ratio) | (FA-aa) | FA-aa (mg/g) | AUC_{no enh} |
| Sunflower oil:Span80 | C12-Leu | 30 | 14 |
| (40:60) | | | |
| Sunflower oil:Span80 | C12-Pro | 30 | 5 |
| (40:60) | | | |
| Sesame oil:Tween80 | C12-Leu | 30 | 7 |
| (30:70) | | | |
| Sesame oil:Span80 | C16-Sarc | 30 | 11 |
| (30:70) | | | |
| Sesame oil:Tween80 | C12-Leu | 30 | 9 |
| (40:60) | | | |
| Sesame oil:Tween80 | C12-Pro | 30 | 13 |
| (40:60) | | | |
| Sesame oil:Tween80 | C12-Trp | 30 | 29 |
| (40:60) | | | |

In conclusion the FA-aa's are functional as absorption enhancers of GH also in non-aqueous formulations.

### Example 16

Non-aqueous formulations of growth hormone containing fatty acid acylated amino acids and a protease inhibitor were tested. A mixture of diglycerol monocaprylate, Tween 20 and propylene glycol (45 wt %, 30 wt %, 15 wt %, 10% water) is used as solvent for growth hormone compound 2 in presence of fatty acid acylated amino acids and protease inhibitors. The composition (150 nmol/rat) was injected into mid-jejunum of anaesthetized overnight fasted Sprague-Dawley rats (n=9) and the pharmacokinetic profile was retrieved and the degree of enhancement calculated as described in example 8. The results are shown in Table H.

TABLE H

| | | | Concentration | |
|---|---|---|---|---|
| | Fatty | Concentration | of protease | |
| Protease | acid amino | of FA-aa | inhibitor | AUCₑₙₕ/ |
| inhibitor | acid (FA-aa) | (mg/g) | mg/g | AUC_{no enh} |
| SBTI | C16-Sarc | 30 | 20 | 23 |
| BBI | C16-Sarc | 30 | 20 | 29 |
| Chymostatin | C16-Sarc | 30 | 20 | 5 |

The data demonstrate that other protease inhibitors than SBTI can be effective in potentiating the effect of the FA-aa.

### Example 17

Aqueous growth hormone formulations containing fatty acid acylated amino acids were tested. Growth hormone compound GH-A2 (129 nmol/rat) dissolved in 10 mM sodium phosphate buffer, pH 8.2 in presence of fatty acid acylated amino acids. The composition was injected into mid-jejunum of anaesthetized overnight fasted Sprague-Dawley rats and the pharmacokinetic profile was retrieved and the degree of enhancement calculated as described in example 8. AUC values are average values for all tested animals. The results are shown in Table I.

TABLE I

| Fatty acid amino | Concentration | | | Number of |
|---|---|---|---|---|
| acid | of FA-aa | Protease | AUCₑₙₕ/ | dosed |
| (FA-aa) | (mg/ml) | inhibitor | AUC_{no enh} | animals |
| C12-Leu | 30 | no | 7 | 12 |
| C16-Glu | 30 | no | 197 | 48 |

### Example 18

Aqueous growth hormone formulations containing fatty acid acylated amino acids and protease inhibitor were tested. Growth hormone compound GH-A2 (129 nmol/rat) dissolved in 100 mM potassium phosphate buffer, pH 8.2 in presence of fatty acid acylated amino acids. The composition was injected into mid-jejunum of anaesthetized overnight fasted Sprague-Dawley rats and the pharmacokinetic profile was retrieved and the degree of enhancement calculated as described in example 8. The AUC values are average values for all tested animals. The results are shown in Table J.

TABLE J

| Fatty acid | Concentration | | | |
|---|---|---|---|---|
| amino acid | of FA-aa | Protease | AUCₑₙₕ/ | Number of |
| (FA-aa) | (mg/ml) | inhibitor | AUC_{no enh} | dosed animals |
| C16-Sarc | 30 | 2% SBTI | 35 | 24 |
| C16-Sarc | 30 | 4% SBTI | 78 | 12 |
| C16-Sarc | 30 | 8% SBTI | 53 | 12 |
| C16-Glu | 30 | 2% SBTI | 61 | 12 |
| C16-Glu | 30 | 4% SBTI | 91 | 12 |
| C16-Glu | 30 | 8% SBTI | 52 | 12 |
| C16-Sarc | 30 | 2% BBI | 17 | 12 |
| C16-Glu | 30 | 2% BBI | 209 | 12 |
| C16-Glu | 30 | 4% BBI | 46 | 12 |
| C16-Glu | 30 | 5% BBI | 40 | 12 |
| C16-Glu | 30 | 10% BBI | 92 | 12 |
| C16-Glu | 30 | no | 88 | 36 |
| C16-Glu | 60 | no | 123 | 12 |
| C16-D-Glu | 30 | no | 35 | 12 |
| C16-Gln | 30 | no | 14 | 12 |
| C12-Trp | 30 | no | 3 | 12 |

### Example 19

Aqueous formulations containing fatty acid acylated amino acids with and without protease inhibitor were tested. Human growth hormone (129 nmol/rat) was dissolved in 100 mM potassium phosphate buffer, pH 8.2 in presence of fatty acid acylated amino acids. The compositions, with or without inhibitor were injected into mid-jejunum of anaesthetized overnight fasted Sprague-Dawley rats and the pharmacokinetic profiles were retrieved and the degree of enhancement calculated. The results are shown in Table K. AUC values are average values for all tested animals.

TABLE K

| | Concentration | | | Number of |
|---|---|---|---|---|
| Fatty acid amino | of FA-aa | Protease | AUCₑₙₕ/ | dosed |
| acid (FA-aa) | (mg/ml) | inhibitor | AUC_{no enh} | animals |
| C16-Glu | 30 | no | 3 | 12 |
| C16-Glu | 30 | 4% SBTI | 20 | 12 |
| C16-Glu | 30 | 8% SBTI | 16 | 12 |

### Example 20

Transepithelial transport of GH compounds in aqueous formulations containing fatty acid acylated amino acids was measured in an E12 monolayer assay.

The transport study was performed as described in General Method D (Example 1). The test was initiated by adding 400 pl test solution (100 μM of GH A1 compound, 0.5 mM fatty acid acylated amino acids and 0.4 μCi/μl [3H]mannitol in transport buffer) to the donor chamber and 1000 pl transport buffer to the receiver chamber. The transport buffer consisting of Hank's balanced saline solution containing 10 mM HEPES, 0.1% was adjusted to pH 7.4 after addition of GH-A1 or GH-A2 compounds. The permeation (Papp) of a Growth hormone compound in the presence of different FA-aa's was measured and the fold increase (or decrease) compared to the Growth hormone compound alone was calculated.

Table L includes the results of tested combinations of GH-A1 and a variety of fatty acid acylated amino acid.

TABLE L

| GH A1 + FA-aa | Fold increase |
|---|---|
| — | 1 |
| C12-Glu | 0.6 |
| C10-Leu | 0.6 |
| C14-D Glu | 0.7 |
| C10-Gln | 0.8 |
| C10-Gly | 0.9 |
| C8-Leu | 1.2 |
| C14-Sarc | 2.4 |
| C12-Trp | 3.5 |
| C14-Gln | 4.5 |
| C16-Gln | 5.1 |
| C16-Glu | 7.0 |

Based on the fold increase relative to GH A1 alone, the combination with C16-Gln or C16-Glu was shown to have the biggest effect on GH A1 transport, whereas as use of C14-aa and a C12 FA-aa have an intermediate effect.

In a further series of permeation experiments transportation of GH-A2 was evaluated using the same procedure as described above. Table M includes the results of tested combinations of GH-A2 and a variety of fatty acid acylated amino acid showing the fold increase relative to GH A2 alone.

TABLE M

| GH A2 + FA-aa | Fold increase |
|---|---|
| C16-Glu | 14.5 |
| C16-Gln | 14.8 |
| C12-Tyr | 9.4 |
| C12-Trp | 9.0 |
| C12-Phe | 8.2 |
| C12-Ile | 8.1 |
| C16-Sarc | 6.5 |
| C14-Gln | 6.1 |
| C14-Leu | 5.9 |
| C14-Sarc | 5.5 |
| C12-Leu | 4.1 |
| C16-(D)-Glu | 4.1 |
| C12-Asp | 3.9 |
| C12-Glu | 3.6 |
| C12-Gly | 3.4 |
| C14-Val | 3.2 |
| C14-Glu | 3.2 |
| C12-Pro | 2.8 |
| C14-(D)-Glu | 2.5 |
| C12-Cys | 2.1 |
| C12-His | 1.9 |
| C12-Ser | 1.9 |
| C16-(D)-Asp | 1.8 |
| C10-Trp | 1.3 |
| C10-Phe | 1.3 |
| C10-Ser | 1.1 |
| C10-Leu | 1.1 |
| C10-Val | 1.1 |
| C10-Tyr | 1.0 |
| C10-Ile | 0.9 |
| C10-Ala | 0.9 |
| C8-Tyr | 0.9 |
| C10-Gly | 0.9 |
| C10-Asp | 0.8 |
| C10-Gln | 0.8 |
| C8-Leu | 0.8 |
| C10-Sarc | 0.7 |
| C8-Ala | 0.7 |
| C10-His | 0.7 |
| C8-Sarc | 0.6 |

As above, the combinations with C16-Gln or C16-Glu were shown to have the biggest effect, while C14- and C12 FA-aa have moderate effect and the C10- and C12 FA-aa have almost no effect on the transportation of GH A2.

The results are thus similar for GH-A1 and GH-A2 with demonstrating that C16-Glu and C16-Gln have superior effect on permeation of the growth hormone compound. C14 and C12 fatty acid amino acid have an intermediate effect on the growth hormone compounds, whereas the C8 and C10 have a very modest, if any effect, on trans-epithelia transport of the growth hormone compounds.

The in vitro data are found to fairly be consistent with the in vivo data: showing that permeation enhancement depends on fatty acid chain length, and to some degree the amino acid residue of the Fatty acid amino acid.

1. A pharmaceutical composition comprising
a. a growth hormone compound and
b. at least one fatty acid amino acid (FA-aa) or a salt thereof, wherein the fatty acid moiety of the FA-aa has 12-18 carbon atoms.

2. The pharmaceutical composition according to claim 1, wherein the composition is an oral pharmaceutical composition.

3. The pharmaceutical composition according to claim 1, wherein the at least one fatty acid amino acid (FA-aa) is of the general formula: 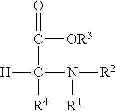
wherein
R1 is a fatty acid chain comprising 10 to 18 carbon atoms,
R2 is H (i.e. hydrogen), CH3 (i.e. methyl group) or a valence bond when R2 is covalently attached to R4, and
R3 is H or absent, and
R4 is an amino acid side chain, including —(CH₂)₃—, when covalently attached to R2.

4. The pharmaceutical composition according to claim 1, wherein the fatty acid moiety of the FA-aa has at least 14 carbon atoms.

5. The pharmaceutical composition according to claim 1, wherein the fatty acid moiety of the FA-aa has up to 16 carbon atoms.

6. The oral pharmaceutical composition according to claim 2, wherein the fatty acid moiety is palmitoyl derived from palmitic acid.

7. The pharmaceutical composition according to claim 1, wherein the amino acid residue of said FA-aa is based on an amino acid selected from the group consisting of: Aspartic acid (Asp), Glutamic acid (Glu), Sarcosine (Sarc), Glycine (Gly), Glutamine (Gln), Asparagine (Asn), Serine (Ser) and Threonine (Thr).

8. The pharmaceutical composition according to claim 7, wherein the amino acid residue is glutamine, glutamic acid or sarcosine.

9. The pharmaceutical composition according to claim 1, wherein the fatty acid acylated amino acid is N-palmitoyl-glutamic acid, sodium or N-palmitoyl-sarcosinate, sodium.

10. The pharmaceutical composition according to claim 1, wherein the growth hormone compound has a T ½ above 30 minutes.

11. A method for increasing bioavailability of a growth hormone compound comprising a step of including a FA-aa in a pharmaceutical composition of said growth hormone compound.

12. A method for increasing the plasma concentration of a growth hormone compound comprising a step of exposing the gastrointestinal tract of an individual to a pharmaceutical composition comprising a growth hormone compound and a FA-aa resulting in an increased plasma concentration of said growth hormone compound in said individual.

13. A method for increasing the up-take of a growth hormone compound comprising the step of: exposing the gastrointestinal tract of an individual to a growth hormone compound and at least one FA-aa, whereby the plasma concentration of said growth hormone in said individual is increased compared to an exposure not including the at least one FA-aa.

14. A method for treatment of a growth hormone related disease or disorder, comprising administering a pharmaceutical composition according to claim 1.

15. The method of claim 11 wherein the fatty acid moiety of the FA-aa in the pharmaceutical composition has 12-18 carbon atoms.

16. A method for treatment of a growth hormone related disease or disorder, comprising administering a pharmaceutical composition according to claim 3.
